(19) United States
(12) Patent Application Publication
(10) Pub. No.: US 2024/0353398 A1
(43) Pub. Date: Oct. 24, 2024

(54) DIAGNOSTIC ASSAY REAGENTS COMPRISING BLOCK CO-POLYMERS

(71) Applicant: OSLER DIAGNOSTICS LIMITED, Oxford (GB)

(72) Inventors: Konstantinos PSARROS, Oxford (GB); Peter READER, Oxford (GB); Sean Edward GOODWIN, Oxford (GB); Thurein THANT, Oxford (GB); Maryam Noor ALI, Oxford (GB); Madiha Fatima DILPAZIR, Oxford (GB); Olivia JONES, Oxford (GB); Fatiha MEDJAHED, Oxford (GB)

(21) Appl. No.: 18/682,753

(22) PCT Filed: Aug. 11, 2022
(86) PCT No.: PCT/EP2022/072601

§ 371 Date: Feb. 9, 2024

(30) Foreign Application Priority Data
Aug. 11, 2021 (GR) 20210100549
Mar. 14, 2022 (GB) 2203536.4

(51) Int. Cl. G01N 33/53; G01N 33/543; G01N 33/68

(52) U.S. Cl. CPC G01N 33/5306; G01N 33/54333; G01N 33/6887; G01N 2333/4712

(57) ABSTRACT

The present invention relates to compositions suitable for use in immunoassays and method of conducting immunoassays using compositions of the invention. The present invention relates to compositions comprising block co-polymers and/or chaotropic agents which are useful as buffer, diluent and/or washing solutions in immunoassays and immunoassay methods.

The present invention relates to compositions suitable for use in immunoassays and method of conducting immunoassays using compositions of the invention. The present invention relates to compositions comprising block co-polymers and/or chaotropic agents which are useful as buffer, diluent and/or washing solutions in immunoassays and immunoassay methods.

### BACKGROUND

Diagnostic tests, such as immunoassays, are often used for the detection of a specific analyte within a sample. For example, pairs of antibodies that can bind to an analyte to form a sandwich that is detectable by means of an enzyme or label on one or more of the antibodies are well known and available for a wide range of different analytes of interest. Antibodies to a particular biomarker, such as testosterone or cortisol, may be used to test levels of these substances in saliva, blood or urine samples.

The presence of the analyte is then determined using, for example, electrochemical measurements or optical measurements. Many electrochemical measurement techniques are known to the skilled person such electrochemical impedance spectroscopy, differential pulse voltammetry, square wave voltammetry, cyclic voltammetry, chronoamperometry, open circuit potential measurement and chronopotentiometry. Optical detection methods can include fluorescence, chemiluminescence, absorbance and surface plasmon resonance, etc. Other suitable detection techniques can be used to analyse experimental outcomes in a sensitive and scalable way. The most common methods are optical detection techniques, electrochemical detection, mechanical analysis, spectroscopy methods (Raman spectroscopy, NMR spectroscopy) and mass spectrometry (MS). All such techniques can be combined with microfluidic devices.

Point-of-care detection brings a diagnostic test conveniently and immediately to a subject, allowing better and faster clinical decisions to be made. However, integration of diagnostic tests into a point-of-care device or system is challenging. Preparation of a sample for an immunoassay may require mixing of multiple solutions and compositions, with precise control of volumes and mixing times. Point-of-care systems also require very fast binding kinetics to enable the typical short test times (<15 min). Further, the device is ideally automated to obviate the need for a medical professional to be present.

Existing liquid handling devices typically flow multiple liquids (such as sample liquids, compositions or wash buffers) across measurement chambers, reaction zones or other detection means in the same flow direction (i.e. different liquids are flowed through the same conduits and parts of the device sequentially). Alternatively, suitable compositions can be used in traditional, laboratory-based devices, such as multiwell plates (e.g. 384- or 96-well plates).

Current compositions used in diagnostic immunoassays can be affected by matrix effects or unspecific reactions and/or can generate significant background signals, thereby affecting the sensitivity of an assay.

Thus, there is a need to provide improved diagnostic assay compositions for use in point-of-care immunoassay diagnostic tests. In particular, there is a need to provide stable compositions that minimise background signal, reduce variation between biological samples and/or improve stability, accuracy and reproducibility of the immunoassay.

### SUMMARY OF THE INVENTION

This summary introduces concepts that are described in more detail in the detailed description. It should not be used to identify essential features of the claimed subject matter, nor to limit the scope of the claimed subject matter.

The present invention provides a composition for immunoassays comprising a buffer compound, a salt and one or more block copolymers.

In some embodiments, the buffer compound is selected from the list consisting of: Tris (tris(hydroxymethyl)aminomethane or 2-amino-2-(hydroxymethyl) propane-1,3-diol), TAPS ([tris(hydroxymethyl)methylamino]propanesulfonic acid), bicine (2-(bis(2-hydroxyethyl)amino) acetic acid), tricine (N-[tris(hydroxymethyl)methyl]glycine), TAPSO (3-[N-tris(hydroxymethyl)methylamino]-2-hydroxypropanesulfonic acid), HEPES (4-(2-hydroxyethyl)-1-piperazineethanesulfonic acid), TES (2-[[1,3-dihydroxy-2-(hydroxymethyl) propan-2-yl]amino]ethanesulfonic acid), MOPS (3-(N-morpholino) propanesulfonic acid), PIPES (piperazine-N,N′-bis(2-ethanesulfonic acid)), cacodylate (dimethylarsenic acid) and MES (2-(N-morpholino) ethanesulfonic acid).

In some embodiments, the salt is selected from the list consisting of: sodium chloride, aluminium ammonium sulphate, aluminium chloride, aluminium chloride, aluminium fluoride, aluminium nitrate, aluminium potassium sulphate, aluminium sulphate, antimony (III) sulphide, antimony trichloride, barium bromide, barium carbonate, barium chloride, barium chromate, barium nitrate, barium perchlorate, barium sulphate, bismuth carbonate, bismuth nitrate, bismuth oxychloride, bismuth subnitrate, bismuth sulphate, bismuth trichloride, cadmium carbonate, cadmium chloride, cadmium iodide, cadmium nitrate, cadmium oxide, cadmium sulphate, calcium bromide, calcium carbonate, calcium chloride, calcium fluoride, calcium nitrate, calcium oxide powder, calcium sulphate, chromium (III) chloride, chromium (III) nitrate, chromium (III) oxide, chromium piconilate, chromium trioxide, cobalt (II) carbonate, cobalt (II) chloride, cobalt (II) nitrate, cobalt (II) sulphate, cobalt oxide, cupric bromide, cupric carbonate, cupric chloride, cupric nitrate, cupric oxide powder, cupric sulphate, cuprous bromide, cuprous chloride, cuprous iodide, cuprous oxide, dysprosium oxide, europium oxide, ferric chloride, ferric nitrate, ferric oxide, ferric sulphate, ferrous chloride, ferrous sulphate, gadolinium (III) oxide, germanium dioxide, holmium oxide, indium (III) chloride, iridium trichloride, lanthanum carbonate, lanthanum chloride, lanthanum nitrate, lanthanum oxalate, lanthanum oxide, lead (II) chloride, lead acetate, lead bromide, lead carbonate, lead dioxide, lead monoxide, lead nitrate, lead oxide, lead sulphate, magnesium bromide, magnesium nitrate, magnesium oxide, magnesium sulphate, manganese (II) sulphate, manganese carbonate, manganese dioxide, mercuric iodide, mercuric nitrate, mercuric oxide, molybdenum trioxide, neodymium oxide, nickel carbonate, nickel chloride, nickel nitrate, nickel oxide, nickel sulphate, niobium pentoxide, platinum chloride, potassium bromide, potassium carbonate, potassium chloride, potassium chloroplatinate, potassium chromate, potassium iodate, potassium iodide, potassium nitrate, potassium pyroantimonate, rhodium trichloride, rubidium chloride, samarium oxide, selenium dioxide, silver benzoate, silver bromide, silver carbonate, silver chloride, silver iodide, silver nitrate, silver oxide, silver sulphate, sodium arsenate, sodium arsenite, sodium bromide, sodium carbonate, sodium fluoride, sodium iodide, sodium metaborate, sodium oxalate, sodium silicate, sodium silicofluoride, sodium stannate, sodium tellurite, stannic chloride, stannic oxide, stannous chloride, stannous sulphate, strontium carbonate, titanium tetrachloride, tungsten (VI) oxide, vanadium pentoxide, yttrium oxide, zinc chloride, zinc cyanide, zinc nitrate, zinc oxide, zinc sulphate, zirconium dioxide and zirconyl nitrate.

In some embodiments, the buffer compound is at a concentration of about 1 mM to about 500 mM, for example about 1 mM to about 450 mM, about 1 mM to about 400 mM, about 1 mM to about 350 mM, about 1 mM to about 300 mM, about 1 mM to about 250 mM, about 1 mM to about 200 mM, about 1 mM to about 150 mM, about 1 mM to about 100 mM, about 1 mM to about 90 mM, about 1 mM to about 80 mM, about 1 mM to about 70 mM, about 1 mM to about 60 mM, about 1 mM to about 50 mM, about 1 mM to about 40 mM, about 1 mM to about 30 mM, about 1 mM to about 20 mM, about 1 mM to about 10 mM, about 10 mM to about 500 mM, about 10 mM to about 450 mM, about 10 mM to about 400 mM, about 10 mM to about 350 mM, about 10 mM to about 300 mM, about 10 mM to about 250 mM, about 10 mM to about 200 mM, about 10 mM to about 150 mM, about 10 mM to about 100 mM, about 10 mM to about 90 mM, about 10 mM to about 80 mM, about 10 mM to about 70 mM, about 10 mM to about 60 mM, about 10 mM to about 50 mM, about 10 mM to about 40 mM, about 10 mM to about 30 mM, about 10 mM to about 20 mM, about 20 mM to about 500 mM, about 20 mM to about 450 mM, about 20 mM to about 400 mM, about 20 mM to about 350 mM, about 20 mM to about 300 mM, about 20 mM to about 250 mM, about 20 mM to about 200 mM, about 20 mM to about 150 mM, about 20 mM to about 100 mM, about 20 mM to about 90 mM, about 20 mM to about 80 mM, about 20 mM to about 70 mM, about 20 mM to about 60 mM, about 20 mM to about 50 mM, about 20 mM to about 40 mM, about 20 mM to about 30 mM, about 30 mM to about 500 mM, about 30 mM to about 450 mM, about 30 mM to about 400 mM, about 30 mM to about 350 mM, about 30 mM to about 300 mM, about 30 mM to about 250 mM, about 30 mM to about 200 mM, about 30 mM to about 150 mM, about 30 mM to about 100 mM, about 30 mM to about 90 mM, about 30 mM to about 80 mM, about 30 mM to about 70 mM, about 30 mM to about 60 mM, about 30 mM to about 50 mM, about 30 mM to about 40 mM, about 40 mM to about 500 mM, about 40 mM to about 450 mM, about 40 mM to about 400 mM, about 40 mM to about 350 mM, about 40 mM to about 300 mM, about 40 mM to about 250 mM, about 40 mM to about 200 mM, about 40 mM to about 150 mM, about 40 mM to about 100 mM, about 40 mM to about 90 mM, about 40 mM to about 80 mM, about 40 mM to about 70 mM, about 40 mM to about 60 mM, about 40 mM to about 50 mM, about 50 mM to about 500 mM, about 50 mM to about 450 mM, about 50 mM to about 400 mM, about 50 mM to about 350 mM, about 50 mM to about 300 mM, about 50 mM to about 250 mM, about 50 mM to about 200 mM, about 50 mM to about 150 mM, about 50 mM to about 100 mM, about 50 mM to about 90 mM, about 50 mM to about 80 mM, about 50 mM to about 70 mM, about 50 mM to about 60 mM, about 60 mM to about 500 mM, about 60 mM to about 450 mM, about 60 mM to about 400 mM, about 60 mM to about 350 mM, about 60 mM to about 300 mM, about 60 mM to about 250 mM, about 60 mM to about 200 mM, about 60 mM to about 150 mM, about 60 mM to about 100 mM, about 60 mM to about 90 mM, about 60 mM to about 80 mM, about 60 mM to about 70 mM, about 70 mM to about 500 mM, about 70 mM to about 450 mM, about 70 mM to about 400 mM, about 70 mM to about 350 mM, about 70 mM to about 300 mM, about 70 mM to about 250 mM, about 70 mM to about 200 mM, about 70 mM to about 150 mM, about 70 mM to about 100 mM, about 70 mM to about 90 mM, about 70 mM to about 80 mM, about 80 mM to about 500 mM, about 80 mM to about 450 mM, about 80 mM to about 400 mM, about 80 mM to about 350 mM, about 80 mM to about 300 mM, about 80 mM to about 250 mM, about 80 mM to about 200 mM, about 80 mM to about 150 mM, about 80 mM to about 100 mM, about 80 mM to about 90 mM, about 90 mM to about 500 mM, about 90 mM to about 450 mM, about 90 mM to about 400 mM, about 90 mM to about 350 mM, about 90 mM to about 300 mM, about 90 mM to about 250 mM, about 90 mM to about 200 mM, about 90 mM to about 150 mM, about 90 mM to about 100 mM, about 100 mM to about 500 mM, about 100 mM to about 450 mM, about 100 mM to about 400 mM, about 100 mM to about 350 mM, about 100 mM to about 300 mM, about 100 mM to about 250 mM, about 100 mM to about 200 mM, about 100 mM to about 150 mM, about 150 mM to about 500 mM, about 150 mM to about 450 mM, about 150 mM to about 400 mM, about 150 mM to about 350 mM, about 150 mM to about 300 mM, about 150 mM to about 250 mM, about 150 mM to about 200 mM, about 200 mM to about 500 mM, about 200 mM to about 450 mM, about 200 mM to about 400 mM, about 200 mM to about 350 mM, about 200 mM to about 300 mM, about 200 mM to about 250 mM, about 250 mM to about 500 mM, about 250 mM to about 450 mM, about 250 mM to about 400 mM, about 250 mM to about 350 mM, about 250 mM to about 300 mM, about 300 mM to about 500 mM, about 300 mM to about 450 mM, about 300 mM to about 400 mM, about 300 mM to about 350 mM, about 350 mM to about 500 mM, about 350 mM to about 450 mM, about 350 mM to about 400 mM, about 400 mM to about 500 mM, about 400 mM to about 450 mM or about 450 mM to about 500 mM.

In some embodiments, the composition comprises Tris (tris(hydroxymethyl)aminomethane or 2-amino-2-(hydroxymethyl) propane-1,3-diol) at a concentration of about 1 mM to about 100 mM, for example about 1 mM to about 90 mM, about 1 mM to about 80 mM, about 1 mM to about 70 mM, about 1 mM to about 60 mM, about 1 mM to about 50 mM, about 1 mM to about 40 mM, about 1 mM to about 30 mM, about 1 mM to about 20 mM, about 1 mM to about 10 mM, about 10 mM to about 100 mM, about 10 mM to about 90 mM, about 10 mM to about 80 mM, about 10 mM to about 70 mM, about 10 mM to about 60 mM, about 10 mM to about 50 mM, about 10 mM to about 40 mM, about 10 mM to about 30 mM, about 10 mM to about 20 mM, about 20 mM to about 100 mM, about 20 mM to about 90 mM, about 20 mM to about 80 mM, about 20 mM to about 70 mM, about 20 mM to about 60 mM, about 20 mM to about 50 mM, about 20 mM to about 40 mM, about 20 mM to about 30 mM, about 30 mM to about 100 mM, about 30 mM to about 90 mM, about 30 mM to about 80 mM, about 30 mM to about 70 mM, about 30 mM to about 60 mM, about 30 mM to about 50 mM, about 30 mM to about 40 mM, about 40 mM to about 100 mM, about 40 mM to about 90 mM, about 40 mM to about 80 mM, about 40 mM to about 70 mM, about 40 mM to about 60 mM, about 40 mM to about 50 mM, about 50 mM to about 100 mM, about 50 mM to about 90 mM, about 50 mM to about 80 mM, about 50 mM to about 70 mM, about 50 mM to about 60 mM, about 60 mM to about 100 mM, about 60 mM to about 90 mM, about 60 mM to about 80 mM, about 60 mM to about 70 mM, about 70 mM to about 100 mM, about 70 mM to about 90 mM, about 70 mM to about 80 mM, about 80 mM to about 100 mM, about 80 mM to about 90 mM, or about 90 mM to about 100 mM.

In some embodiments, the composition comprises Tris (tris(hydroxymethyl)aminomethane or 2-amino-2-(hydroxymethyl) propane-1,3-diol) at a concentration of about 10 mM to 100 mM. In some embodiments, the composition comprises Tris (tris(hydroxymethyl)aminomethane or 2-amino-2-(hydroxymethyl) propane-1,3-diol) at a concentration of about 20 mM to 30 mM. In some embodiments, the composition comprises Tris (tris(hydroxymethyl)aminomethane or 2-amino-2-(hydroxymethyl) propane-1,3-diol) at a concentration of 25 mM.

In some embodiments, the salt is at a concentration of about 1 mM to about 1000 mM, for example about 1 mM to about 900 mM, about 1 mM to about 800 mM, about 1 mM to about 700 mM, about 1 mM to about 600 mM, about 1 mM to about 500 mM, about 1 mM to about 400 mM, about 1 mM to about 300 mM, about 1 mM to about 200 mM, about 1 mM to about 100 mM, about 1 mM to about 50 mM, about 50 mM to about 1000 mM, about 50 mM to about 900 mM, about 50 mM to about 800 mM, about 50 mM to about 700 mM, about 50 mM to about 600 mM, about 50 mM to about 500 mM, about 50 mM to about 400 mM, about 50 mM to about 300 mM, about 50 mM to about 200 mM, about 50 mM to about 100 mM, about 100 mM to about 1000 mM, about 100 mM to about 900 mM, about 100 mM to about 800 mM, about 100 mM to about 700 mM, about 100 mM to about 600 mM, about 100 mM to about 500 mM, about 100 mM to about 400 mM, about 100 mM to about 300 mM, about 100 mM to about 200 mM, about 200 mM to about 1000 mM, about 200 mM to about 900 mM, about 200 mM to about 800 mM, about 200 mM to about 700 mM, about 200 mM to about 600 mM, about 200 mM to about 500 mM, about 200 mM to about 400 mM, about 200 mM to about 300 mM, about 300 mM to about 1000 mM, about 300 mM to about 900 mM, about 300 mM to about 800 mM, about 300 mM to about 700 mM, about 300 mM to about 600 mM, about 300 mM to about 500 mM, about 300 mM to about 400 mM, about 400 mM to about 1000 mM, about 400 mM to about 900 mM, about 400 mM to about 800 mM, about 400 mM to about 700 mM, about 400 mM to about 600 mM, about 400 mM to about 500 mM, about 500 mM to about 1000 mM, about 500 mM to about 900 mM, about 500 mM to about 800 mM, about 500 mM to about 700 mM, about 500 mM to about 600 mM, about 600 mM to about 1000 mM, about 600 mM to about 900 mM, about 600 mM to about 800 mM, about 600 mM to about 700 mM, about 700 mM to about 1000 mM, about 700 mM to about 900 mM, about 700 mM to about 800 mM, about 800 mM to about 1000 mM, about 800 mM to about 900 mM or about 900 mM to about 1000 mM.

In some embodiments, the composition comprises sodium chloride (NaCl) at a concentration of about 1 mM to about 1000 mM, for example about 1 mM to about 900 mM, about 1 mM to about 800 mM, about 1 mM to about 700 mM, about 1 mM to about 600 mM, about 1 mM to about 500 mM, about 1 mM to about 400 mM, about 1 mM to about 300 mM, about 1 mM to about 200 mM, about 1 mM to about 100 mM, about 1 mM to about 50 mM, about 50 mM to about 1000 mM, about 50 mM to about 900 mM, about 50 mM to about 800 mM, about 50 mM to about 700 mM, about 50 mM to about 600 mM, about 50 mM to about 500 mM, about 50 mM to about 400 mM, about 50 mM to about 300 mM, about 50 mM to about 200 mM, about 50 mM to about 100 mM, about 100 mM to about 1000 mM, about 100 mM to about 900 mM, about 100 mM to about 800 mM, about 100 mM to about 700 mM, about 100 mM to about 600 mM, about 100 mM to about 500 mM, about 100 mM to about 400 mM, about 100 mM to about 300 mM, about 100 mM to about 200 mM, about 200 mM to about 1000 mM, about 200 mM to about 900 mM, about 200 mM to about 800 mM, about 200 mM to about 700 mM, about 200 mM to about 600 mM, about 200 mM to about 500 mM, about 200 mM to about 400 mM, about 200 mM to about 300 mM, about 300 mM to about 1000 mM, about 300 mM to about 900 mM, about 300 mM to about 800 mM, about 300 mM to about 700 mM, about 300 mM to about 600 mM, about 300 mM to about 500 mM, about 300 mM to about 400 mM, about 400 mM to about 1000 mM, about 400 mM to about 900 mM, about 400 mM to about 800 mM, about 400 mM to about 700 mM, about 400 mM to about 600 mM, about 400 mM to about 500 mM, about 500 mM to about 1000 mM, about 500 mM to about 900 mM, about 500 mM to about 800 mM, about 500 mM to about 700 mM, about 500 mM to about 600 mM, about 600 mM to about 1000 mM, about 600 mM to about 900 mM, about 600 mM to about 800 mM, about 600 mM to about 700 mM, about 700 mM to about 1000 mM, about 700 mM to about 900 mM, about 700 mM to about 800 mM, about 800 mM to about 1000 mM, about 800 mM to about 900 mM or about 900 mM to about 1000 mM. In some embodiments, the composition comprises sodium chloride (NaCl) at a concentration of about 50 to about 500 mM. In some embodiments, the composition comprises sodium chloride (NaCl) at a concentration of about 200 to about 400 mM. In some embodiments, the composition comprises sodium chloride (NaCl) at a concentration of about 100 to about 200 mM.

In some embodiments, the composition comprises sodium chloride (NaCl) at a concentration of 150 mM. In some embodiments, the composition comprises sodium chloride (NaCl) at a concentration of 400 mM. In some embodiments, the composition comprises sodium chloride (NaCl) at a concentration of about 275 mM.

In some embodiments, the composition comprises Tris (tris(hydroxymethyl)aminomethane or 2-amino-2-(hydroxymethyl) propane-1,3-diol), sodium chloride and one or more block copolymers.

In some embodiments, the composition comprises tris-buffered saline (TBS). In some embodiments, the tris-buffered saline (TBS) comprises Tris (tris(hydroxymethyl)aminomethane or 2-amino-2-(hydroxymethyl) propane-1,3-diol) at a concentration of about 20 mM to 30 mM and comprises NaCl at a concentration of about 100 mM to 200 mM. In some embodiments, the tris-buffered saline (TBS) comprises Tris (tris(hydroxymethyl)aminomethane or 2-amino-2-(hydroxymethyl) propane-1,3-diol) at a concentration of 25 mM and comprises NaCl at a concentration of 150 mM. In some embodiments, the tris-buffered saline (TBS) comprises Tris (tris(hydroxymethyl)aminomethane or 2-amino-2-(hydroxymethyl) propane-1,3-diol) at a concentration of about 20 mM to 30 mM and comprises NaCl at a concentration of about 50 mM to 500 mM. In some embodiments, the tris-buffered saline (TBS) comprises Tris (tris(hydroxymethyl)aminomethane or 2-amino-2-(hydroxymethyl) propane-1,3-diol) at a concentration of 25 mM and comprises NaCl at a concentration of 400 mM.

In some embodiments, the composition comprises tris-buffered saline (TBS), which comprises Tris (tris(hydroxymethyl) aminomethane or 2-amino-2-(hydroxymethyl) propane-1,3-diol) and NaCl, and additional NaCl. The NaCl from the TBS and the additional NaCl, combined, provide the NaCl concentration in the composition. In some embodiments, the composition comprises Tris (tris(hydroxymethyl) aminomethane or 2-amino-2-(hydroxymethyl) propane-1,3-diol) at a concentration of about 20 mM to about 30 mM and NaCl at a concentration of about 200 mM to about 500 mM. In some embodiments, the composition comprises Tris (tris(hydroxymethyl) aminomethane or 2-amino-2-(hydroxymethyl) propane-1,3-diol) at a concentration of about 20 mM to about 30 mM and NaCl at a concentration of about 200 mM to about 400 mM. In some embodiments, the composition comprises Tris (tris(hydroxymethyl) aminomethane or 2-amino-2-(hydroxymethyl) propane-1,3-diol) at a concentration of about 25 mM and NaCl at a concentration of about 275 mM.

In some embodiments, the composition comprises one or more block copolymers at a total concentration of about 0.01% (w/v) to about 2% (w/v), for example about 0.01% (w/v) to about 1.9% (w/v), about 0.01% (w/v) to about 1.8% (w/v), about 0.01% (w/v) to about 1.7% (w/v), about 0.01% (w/v) to about 1.6% (w/v), about 0.01% (w/v) to about 1.5% (w/v), about 0.01% (w/v) to about 1.4% (w/v), about 0.01% (w/v) to about 1.3% (w/v), about 0.01% (w/v) to about 1.2% (w/v), about 0.01% (w/v) to about 1.1% (w/v), about 0.01% (w/v) to about 1% (w/v), about 0.01% (w/v) to about 0.9% (w/v), about 0.01% (w/v) to about 0.8% (w/v), about 0.01% (w/v) to about 0.7% (w/v), about 0.01% (w/v) to about 0.6% (w/v), about 0.01% (w/v) to about 0.5% (w/v), about 0.01% (w/v) to about 0.4% (w/v), about 0.01% (w/v) to about 0.3% (w/v), about 0.01% (w/v) to about 0.2% (w/v), about 0.01% (w/v) to about 0.1% (w/v), about 0.01% (w/v) to about 0.05% (w/v), about 0.05% (w/v) to about 2% (w/v), about 0.05% (w/v) to about 1.9% (w/v), about 0.05% (w/v) to about 1.8% (w/v), about 0.05% (w/v) to about 1.7% (w/v), about 0.05% (w/v) to about 1.6% (w/v), about 0.05% (w/v) to about 1.5% (w/v), about 0.05% (w/v) to about 1.4% (w/v), about 0.05% (w/v) to about 1.3% (w/v), about 0.05% (w/v) to about 1.2% (w/v), about 0.05% (w/v) to about 1.1% (w/v), about 0.05% (w/v) to about 1% (w/v), about 0.05% (w/v) to about 0.9% (w/v), about 0.05% (w/v) to about 0.8% (w/v), about 0.05% (w/v) to about 0.7% (w/v), about 0.05% (w/v) to about 0.6% (w/v), about 0.05% (w/v) to about 0.5% (w/v), about 0.05% (w/v) to about 0.4% (w/v), about 0.05% (w/v) to about 0.3% (w/v), about 0.05% (w/v) to about 0.2% (w/v), about 0.05% (w/v) to about 0.1% (w/v), about 0.1% (w/v) to about 2% (w/v), about 0.1% (w/v) to about 1.9% (w/v), about 0.1% (w/v) to about 1.8% (w/v), about 0.1% (w/v) to about 1.7% (w/v), about 0.1% (w/v) to about 1.6% (w/v), about 0.1% (w/v) to about 1.5% (w/v), about 0.1% (w/v) to about 1.4% (w/v), about 0.1% (w/v) to about 1.3% (w/v), about 0.1% (w/v) to about 1.2% (w/v), about 0.1% (w/v) to about 1.1% (w/v), about 0.1% (w/v) to about 1% (w/v), about 0.1% (w/v) to about 0.9% (w/v), about 0.1% (w/v) to about 0.8% (w/v), about 0.1% (w/v) to about 0.7% (w/v), about 0.1% (w/v) to about 0.6% (w/v), about 0.1% (w/v) to about 0.5% (w/v), about 0.1% (w/v) to about 0.4% (w/v), about 0.1% (w/v) to about 0.3% (w/v), about 0.1% (w/v) to about 0.2% (w/v), about 0.2% (w/v) to about 2% (w/v), about 0.2% (w/v) to about 1.9% (w/v), about 0.2% (w/v) to about 1.8% (w/v), about 0.2% (w/v) to about 1.7% (w/v), about 0.2% (w/v) to about 1.6% (w/v), about 0.2% (w/v) to about 1.5% (w/v), about 0.2% (w/v) to about 1.4% (w/v), about 0.2% (w/v) to about 1.3% (w/v), about 0.2% (w/v) to about 1.2% (w/v), about 0.2% (w/v) to about 1.1% (w/v), about 0.2% (w/v) to about 1% (w/v), about 0.2% (w/v) to about 0.9% (w/v), about 0.2% (w/v) to about 0.8% (w/v), about 0.2% (w/v) to about 0.7% (w/v), about 0.2% (w/v) to about 0.6% (w/v), about 0.2% (w/v) to about 0.5% (w/v), about 0.2% (w/v) to about 0.4% (w/v), about 0.2% (w/v) to about 0.3% (w/v), about 0.3% (w/v) to about 2% (w/v), about 0.3% (w/v) to about 1.9% (w/v), about 0.3% (w/v) to about 1.8% (w/v), about 0.3% (w/v) to about 1.7% (w/v), about 0.3% (w/v) to about 1.6% (w/v), about 0.3% (w/v) to about 1.5% (w/v), about 0.3% (w/v) to about 1.4% (w/v), about 0.3% (w/v) to about 1.3% (w/v), about 0.3% (w/v) to about 1.2% (w/v), about 0.3% (w/v) to about 1.1% (w/v), about 0.3% (w/v) to about 1% (w/v), about 0.3% (w/v) to about 0.9% (w/v), about 0.3% (w/v) to about 0.8% (w/v), about 0.3% (w/v) to about 0.7% (w/v), about 0.3% (w/v) to about 0.6% (w/v), about 0.3% (w/v) to about 0.5% (w/v), about 0.3% (w/v) to about 0.4% (w/v), about 0.4% (w/v) to about 2% (w/v), about 0.4% (w/v) to about 1.9% (w/v), about 0.4% (w/v) to about 1.8% (w/v), about 0.4% (w/v) to about 1.7% (w/v), about 0.4% (w/v) to about 1.6% (w/v), about 0.4% (w/v) to about 1.5% (w/v), about 0.4% (w/v) to about 1.4% (w/v), about 0.4% (w/v) to about 1.3% (w/v), about 0.4% (w/v) to about 1.2% (w/v), about 0.4% (w/v) to about 1.1% (w/v), about 0.4% (w/v) to about 1% (w/v), about 0.4% (w/v) to about 0.9% (w/v), about 0.4% (w/v) to about 0.8% (w/v), about 0.4% (w/v) to about 0.7% (w/v), about 0.4% (w/v) to about 0.6% (w/v), about 0.4% (w/v) to about 0.5% (w/v), about 0.5% (w/v) to about 2% (w/v), about 0.5% (w/v) to about 1.9% (w/v), about 0.5% (w/v) to about 1.8% (w/v), about 0.5% (w/v) to about 1.7% (w/v), about 0.5% (w/v) to about 1.6% (w/v), about 0.5% (w/v) to about 1.5% (w/v), about 0.5% (w/v) to about 1.4% (w/v), about 0.5% (w/v) to about 1.3% (w/v), about 0.5% (w/v) to about 1.2% (w/v), about 0.5% (w/v) to about 1.1% (w/v), about 0.5% (w/v) to about 1% (w/v), about 0.5% (w/v) to about 0.9% (w/v), about 0.5% (w/v) to about 0.8% (w/v), about 0.5% (w/v) to about 0.7% (w/v), about 0.5% (w/v) to about 0.6% (w/v), about 0.6% (w/v) to about 2% (w/v), about 0.6% (w/v) to about 1.9% (w/v), about 0.6% (w/v) to about 1.8% (w/v), about 0.6% (w/v) to about 1.7% (w/v), about 0.6% (w/v) to about 1.6% (w/v), about 0.6% (w/v) to about 1.5% (w/v), about 0.6% (w/v) to about 1.4% (w/v), about 0.6% (w/v) to about 1.3% (w/v), about 0.6% (w/v) to about 1.2% (w/v), about 0.6% (w/v) to about 1.1% (w/v), about 0.6% (w/v) to about 1% (w/v), about 0.6% (w/v) to about 0.9% (w/v), about 0.6% (w/v) to about 0.8% (w/v), about 0.6% (w/v) to about 0.7% (w/v), about 0.7% (w/v) to about 2% (w/v), about 0.7% (w/v) to about 1.9% (w/v), about 0.7% (w/v) to about 1.8% (w/v), about 0.7% (w/v) to about 1.7% (w/v), about 0.7% (w/v) to about 1.6% (w/v), about 0.7% (w/v) to about 1.5% (w/v), about 0.7% (w/v) to about 1.4% (w/v), about 0.7% (w/v) to about 1.3% (w/v), about 0.7% (w/v) to about 1.2% (w/v), about 0.7% (w/v) to about 1.1% (w/v), about 0.7% (w/v) to about 1% (w/v), about 0.7% (w/v) to about 0.9% (w/V), about 0.7% (w/v) to about 0.8% (w/v), about 0.8% (w/v) to about 2% (w/v), about 0.8% (w/v) to about 1.9% (w/v), about 0.8% (w/v) to about 1.8% (w/v), about 0.8% (w/v) to about 1.7% (w/v), about 0.8% (w/v) to about 1.6% (w/v), about 0.8% (w/v) to about 1.5% (w/v), about 0.8% (w/v) to about 1.4% (w/v), about 0.8% (w/v) to about 1.3% (w/v), about 0.8% (w/v) to about 1.2% (w/v), about 0.8% (w/v) to about 1.1% (w/v), about 0.8% (w/v) to about 1% (w/v), about 0.8% (w/v) to about 0.9% (w/v), about 0.9% (w/v) to about 2% (w/v), about 0.9% (w/v) to about 1.9% (w/v), about 0.9% (w/v) to about 1.8% (w/v), about 0.9% (w/v) to about 1.7% (w/v), about 0.9% (w/v) to about 1.6% (w/v), about 0.9% (w/v) to about 1.5% (w/v), about 0.9% (w/v) to about 1.4% (w/v), about 0.9% (w/v) to about 1.3% (w/v), about 0.9% (w/v) to about 1.2% (w/v), about 0.9% (w/v) to about 1.1% (w/v), about 0.9% (w/v) to about 1% (w/v), about 1% (w/v) to about 2% (w/v), about 1% (w/v) to about 1.9% (w/v), about 1% (w/v) to about 1.8% (w/v), about 1% (w/v) to about 1.7% (w/v), about 1% (w/v) to about 1.6% (w/v), about 1% (w/v) to about 1.5% (w/v), about 1% (w/v) to about 1.4% (w/v), about 1% (w/v) to about 1.3% (w/v), about 1% (w/v) to about 1.2% (w/v), about 1% (w/v) to about 1.1% (w/v), about 1.1% (w/v) to about 2% (w/v), about 1.1% (w/v) to about 1.9% (w/v), about 1.1% (w/v) to about 1.8% (w/v), about 1.1% (w/v) to about 1.7% (w/v), about 1.1% (w/v) to about 1.6% (w/v), about 1.1% (w/v) to about 1.5% (w/v), about 1.1% (w/v) to about 1.4% (w/v), about 1.1% (w/v) to about 1.3% (w/v), about 1.1% (w/v) to about 1.2% (w/v), about 1.2% (w/v) to about 2% (w/v), about 1.2% (w/v) to about 1.9% (w/v), about 1.2% (w/v) to about 1.8% (w/v), about 1.2% (w/v) to about 1.7% (w/v), about 1.2% (w/v) to about 1.6% (w/v), about 1.2% (w/v) to about 1.5% (w/v), about 1.2% (w/v) to about 1.4% (w/v), about 1.2% (w/v) to about 1.3% (w/v), about 1.3% (w/v) to about 2% (w/v), about 1.3% (w/v) to about 1.9% (w/v), about 1.3% (w/v) to about 1.8% (w/v), about 1.3% (w/v) to about 1.7% (w/v), about 1.3% (w/v) to about 1.6% (w/v), about 1.3% (w/v) to about 1.5% (w/v), about 1.3% (w/v) to about 1.4% (w/v), about 1.4% (w/v) to about 2% (w/v), about 1.4% (w/v) to about 1.9% (w/v), about 1.4% (w/v) to about 1.8% (w/v), about 1.4% (w/v) to about 1.7% (w/v), about 1.4% (w/v) to about 1.6% (w/v), about 1.4% (w/v) to about 1.5% (w/v), about 1.5% (w/v) to about 2% (w/v), about 1.5% (w/v) to about 1.9% (w/v), about 1.5% (w/v) to about 1.8% (w/v), about 1.5% (w/v) to about 1.7% (w/v), about 1.5% (w/v) to about 1.6% (w/v), about 1.6% (w/v) to about 2% (w/v), about 1.6% (w/v) to about 1.9% (w/v), about 1.6% (w/v) to about 1.8% (w/v), about 1.6% (w/v) to about 1.7% (w/v), about 1.7% (w/v) to about 2% (w/v), about 1.7% (w/v) to about 1.9% (w/v), about 1.7% (w/v) to about 1.8% (w/v), about 1.8% (w/v) to about 2% (w/v), about 1.8% (w/v) to about 1.9% (w/v) or about 1.9% (w/v) to about 2% (w/v).

In some embodiments, the composition comprises one or more block copolymers at a total concentration of about 0.25% (w/v) to about 1.5% (w/v). In some embodiments, the composition comprises one or more block copolymers at a total concentration of about 0.05% (w/v). In some embodiments, the composition comprises one or more block copolymers at a total concentration of about 0.125% (w/v). In some embodiments the composition comprises one block copolymer at a total concentration of about 0.05% (w/v). In some embodiments, the composition comprises one block copolymer at a total concentration of about 0.125% (w/v).

In some embodiments, the composition comprises Tris (tris(hydroxymethyl) aminomethane or 2-amino-2-(hydroxymethyl) propane-1,3-diol) at a concentration of about 1 mM to 500 mM, sodium chloride (NaCl) at a concentration of about 1 mM to 1000 mM and one or more block copolymers at a total concentration of about 0.01% (w/v) to 2% (w/v). In some embodiments, the composition comprises Tris (tris(hydroxymethyl) aminomethane or 2-amino-2-(hydroxymethyl) propane-1,3-diol) at a concentration of 25 mM, sodium chloride (NaCl) at a concentration of 150 mM and a block copolymer at a concentration of 0.05% (w/v). In some embodiments, the composition comprises Tris (tris(hydroxymethyl) aminomethane or 2-amino-2-(hydroxymethyl) propane-1,3-diol) at a concentration of 25 mM, sodium chloride (NaCl) at a concentration of 275 mM and a block copolymer at a concentration of 0.125% (w/v).

The present invention also provides a composition for immunoassays comprising a buffer compound, one or more salts, one or more blocking agents and one or more block copolymers. In some embodiments, the one or more blocking agents are selected from the list consisting of: proteins, such as phosphoproteins, polymers and small molecules. In some embodiments, the protein is a phosphoprotein, optionally wherein the phosphoprotein is casein. In some embodiments, the protein is bovine serum albumin (BSA). In some embodiments, the polymer is PVP (polyvinylpyrrolidone) or PVA (polyvinyl alcohol). In some embodiments, the small molecule is ethanolamine.

In some embodiments, the composition comprises at least two salts, for example two, three, four, five or six salts. In some embodiments, the composition comprises two salts.

In some embodiments, the buffer compound is selected from the list consisting of: Tris (tris(hydroxymethyl) aminomethane or 2-amino-2-(hydroxymethyl) propane-1,3-diol), TAPS ([tris(hydroxymethyl) methylamino]propanesulfonic acid), bicine (2-(bis(2-hydroxyethyl)amino) acetic acid), tricine (N-[tris(hydroxymethyl) methyl]glycine), TAPSO (3-[N-tris(hydroxymethyl) methylamino]-2-hydroxypropanesulfonic acid), HEPES (4-(2-hydroxyethyl)-1-piperazineethanesulfonic acid), TES (2-[[1,3-dihydroxy-2-(hydroxymethyl) propan-2-yl]amino]ethanesulfonic acid), MOPS (3-(N-morpholino) propanesulfonic acid), PIPES (piperazine-N,N′-bis(2-ethanesulfonic acid)), cacodylate (dimethylarsenic acid) and MES (2-(N-morpholino) ethanesulfonic acid)

In some embodiments, the one or more salts are selected from the list consisting of: sodium chloride, calcium chloride, aluminium ammonium sulphate, aluminium chloride, aluminium chloride, aluminium fluoride, aluminium nitrate, aluminium potassium sulphate, aluminium sulphate, antimony (III) sulphide, antimony trichloride, barium bromide, barium carbonate, barium chloride, barium chromate, barium nitrate, barium perchlorate, barium sulphate, bismuth carbonate, bismuth nitrate, bismuth oxychloride, bismuth subnitrate, bismuth sulphate, bismuth trichloride, cadmium carbonate, cadmium chloride, cadmium iodide, cadmium nitrate, cadmium oxide, cadmium sulphate, calcium bromide, calcium carbonate, calcium fluoride, calcium nitrate, calcium oxide powder, calcium sulphate, chromium (III) chloride, chromium (III) nitrate, chromium (III) oxide, chromium piconilate, chromium trioxide, cobalt (II) carbonate, cobalt (II) chloride, cobalt (II) nitrate, cobalt (II) sulphate, cobalt oxide, cupric bromide, cupric carbonate, cupric chloride, cupric nitrate, cupric oxide powder, cupric sulphate, cuprous bromide, cuprous chloride, cuprous iodide, cuprous oxide, dysprosium oxide, europium oxide, ferric chloride, ferric nitrate, ferric oxide, ferric sulphate, ferrous chloride, ferrous sulphate, gadolinium (III) oxide, germanium dioxide, holmium oxide, indium (III) chloride, iridium trichloride, lanthanum carbonate, lanthanum chloride, lanthanum nitrate, lanthanum oxalate, lanthanum oxide, lead (II) chloride, lead acetate, lead bromide, lead carbonate, lead dioxide, lead monoxide, lead nitrate, lead oxide, lead sulphate, magnesium bromide, magnesium nitrate, magnesium oxide, magnesium sulphate, manganese (II) sulphate, manganese carbonate, manganese dioxide, mercuric iodide, mercuric nitrate, mercuric oxide, molybdenum trioxide, neodymium oxide, nickel carbonate, nickel chloride, nickel nitrate, nickel oxide, nickel sulphate, niobium pentoxide, platinum chloride, potassium bromide, potassium carbonate, potassium chloride, potassium chloroplatinate, potassium chromate, potassium iodate, potassium iodide, potassium nitrate, potassium pyroantimonate, rhodium trichloride, rubidium chloride, samarium oxide, selenium dioxide, silver benzoate, silver bromide, silver carbonate, silver chloride, silver iodide, silver nitrate, silver oxide, silver sulphate, sodium arsenate, sodium arsenite, sodium bromide, sodium carbonate, sodium fluoride, sodium iodide, sodium metaborate, sodium oxalate, sodium silicate, sodium silicofluoride, sodium stannate, sodium tellurite, stannic chloride, stannic oxide, stannous chloride, stannous sulphate, strontium carbonate, titanium tetrachloride, tungsten (VI) oxide, vanadium pentoxide, yttrium oxide, zinc chloride, zinc cyanide, zinc nitrate, zinc oxide, zinc sulphate, zirconium dioxide and zirconyl nitrate.

In some embodiments, the buffer compound is at a concentration of about 1 mM to about 500 mM, for example about 1 mM to about 450 mM, about 1 mM to about 400 mM, about 1 mM to about 350 mM, about 1 mM to about 300 mM, about 1 mM to about 250 mM, about 1 mM to about 200 mM, about 1 mM to about 150 mM, about 1 mM to about 100 mM, about 1 mM to about 90 mM, about 1 mM to about 80 mM, about 1 mM to about 70 mM, about 1 mM to about 60 mM, about 1 mM to about 50 mM, about 1 mM to about 40 mM, about 1 mM to about 30 mM, about 1 mM to about 20 mM, about 1 mM to about 10 mM, about 10 mM to about 500 mM, about 10 mM to about 450 mM, about 10 mM to about 400 mM, about 10 mM to about 350 mM, about 10 mM to about 300 mM, about 10 mM to about 250 mM, about 10 mM to about 200 mM, about 10 mM to about 150 mM, about 10 mM to about 100 mM, about 10 mM to about 90 mM, about 10 mM to about 80 mM, about 10 mM to about 70 mM, about 10 mM to about 60 mM, about 10 mM to about 50 mM, about 10 mM to about 40 mM, about 10 mM to about 30 mM, about 10 mM to about 20 mM, about 20 mM to about 500 mM, about 20 mM to about 450 mM, about 20 mM to about 400 mM, about 20 mM to about 350 mM, about 20 mM to about 300 mM, about 20 mM to about 250 mM, about 20 mM to about 200 mM, about 20 mM to about 150 mM, about 20 mM to about 100 mM, about 20 mM to about 90 mM, about 20 mM to about 80 mM, about 20 mM to about 70 mM, about 20 mM to about 60 mM, about 20 mM to about 50 mM, about 20 mM to about 40 mM, about 20 mM to about 30 mM, about 30 mM to about 500 mM, about 30 mM to about 450 mM, about 30 mM to about 400 mM, about 30 mM to about 350 mM, about 30 mM to about 300 mM, about 30 mM to about 250 mM, about 30 mM to about 200 mM, about 30 mM to about 150 mM, about 30 mM to about 100 mM, about 30 mM to about 90 mM, about 30 mM to about 80 mM, about 30 mM to about 70 mM, about 30 mM to about 60 mM, about 30 mM to about 50 mM, about 30 mM to about 40 mM, about 40 mM to about 500 mM, about 40 mM to about 450 mM, about 40 mM to about 400 mM, about 40 mM to about 350 mM, about 40 mM to about 300 mM, about 40 mM to about 250 mM, about 40 mM to about 200 mM, about 40 mM to about 150 mM, about 40 mM to about 100 mM, about 40 mM to about 90 mM, about 40 mM to about 80 mM, about 40 mM to about 70 mM, about 40 mM to about 60 mM, about 40 mM to about 50 mM, about 50 mM to about 500 mM, about 50 mM to about 450 mM, about 50 mM to about 400 mM, about 50 mM to about 350 mM, about 50 mM to about 300 mM, about 50 mM to about 250 mM, about 50 mM to about 200 mM, about 50 mM to about 150 mM, about 50 mM to about 100 mM, about 50 mM to about 90 mM, about 50 mM to about 80 mM, about 50 mM to about 70 mM, about 50 mM to about 60 mM, about 60 mM to about 500 mM, about 60 mM to about 450 mM, about 60 mM to about 400 mM, about 60 mM to about 350 mM, about 60 mM to about 300 mM, about 60 mM to about 250 mM, about 60 mM to about 200 mM, about 60 mM to about 150 mM, about 60 mM to about 100 mM, about 60 mM to about 90 mM, about 60 mM to about 80 mM, about 60 mM to about 70 mM, about 70 mM to about 500 mM, about 70 mM to about 450 mM, about 70 mM to about 400 mM, about 70 mM to about 350 mM, about 70 mM to about 300 mM, about 70 mM to about 250 mM, about 70 mM to about 200 mM, about 70 mM to about 150 mM, about 70 mM to about 100 mM, about 70 mM to about 90 mM, about 70 mM to about 80 mM, about 80 mM to about 500 mM, about 80 mM to about 450 mM, about 80 mM to about 400 mM, about 80 mM to about 350 mM, about 80 mM to about 300 mM, about 80 mM to about 250 mM, about 80 mM to about 200 mM, about 80 mM to about 150 mM, about 80 mM to about 100 mM, about 80 mM to about 90 mM, about 90 mM to about 500 mM, about 90 mM to about 450 mM, about 90 mM to about 400 mM, about 90 mM to about 350 mM, about 90 mM to about 300 mM, about 90 mM to about 250 mM, about 90 mM to about 200 mM, about 90 mM to about 150 mM, about 90 mM to about 100 mM, about 100 mM to about 500 mM, about 100 mM to about 450 mM, about 100 mM to about 400 mM, about 100 mM to about 350 mM, about 100 mM to about 300 mM, about 100 mM to about 250 mM, about 100 mM to about 200 mM, about 100 mM to about 150 mM, about 150 mM to about 500 mM, about 150 mM to about 450 mM, about 150 mM to about 400 mM, about 150 mM to about 350 mM, about 150 mM to about 300 mM, about 150 mM to about 250 mM, about 150 mM to about 200 mM, about 200 mM to about 500 mM, about 200 mM to about 450 mM, about 200 mM to about 400 mM, about 200 mM to about 350 mM, about 200 mM to about 300 mM, about 200 mM to about 250 mM, about 250 mM to about 500 mM, about 250 mM to about 450 mM, about 250 mM to about 400 mM, about 250 mM to about 350 mM, about 250 mM to about 300 mM, about 300 mM to about 500 mM, about 300 mM to about 450 mM, about 300 mM to about 400 mM, about 300 mM to about 350 mM, about 350 mM to about 500 mM, about 350 mM to about 450 mM, about 350 mM to about 400 mM, about 400 mM to about 500 mM, about 400 mM to about 450 mM or about 450 mM to about 500 mM.

In some embodiments, the composition comprises Tris (tris(hydroxymethyl) aminomethane or 2-amino-2-(hydroxymethyl) propane-1,3-diol) at a concentration of about 1 mM to about 100 mM, for example about 1 mM to about 90 mM, about 1 mM to about 80 mM, about 1 mM to about 70 mM, about 1 mM to about 60 mM, about 1 mM to about 50 mM, about 1 mM to about 40 mM, about 1 mM to about 30 mM, about 1 mM to about 20 mM, about 1 mM to about 10 mM, about 10 mM to about 100 mM, about 10 mM to about 90 mM, about 10 mM to about 80 mM, about 10 mM to about 70 mM, about 10 mM to about 60 mM, about 10 mM to about 50 mM, about 10 mM to about 40 mM, about 10 mM to about 30 mM, about 10 mM to about 20 mM, about 20 mM to about 100 mM, about 20 mM to about 90 mM, about 20 mM to about 80 mM, about 20 mM to about 70 mM, about 20 mM to about 60 mM, about 20 mM to about 50 mM, about 20 mM to about 40 mM, about 20 mM to about 30 mM, about 30 mM to about 100 mM, about 30 mM to about 90 mM, about 30 mM to about 80 mM, about 30 mM to about 70 mM, about 30 mM to about 60 mM, about 30 mM to about 50 mM, about 30 mM to about 40 mM, about 40 mM to about 100 mM, about 40 mM to about 90 mM, about 40 mM to about 80 mM, about 40 mM to about 70 mM, about 40 mM to about 60 mM, about 40 mM to about 50 mM, about 50 mM to about 100 mM, about 50 mM to about 90 mM, about 50 mM to about 80 mM, about 50 mM to about 70 mM, about 50 mM to about 60 mM, about 60 mM to about 100 mM, about 60 mM to about 90 mM, about 60 mM to about 80 mM, about 60 mM to about 70 mM, about 70 mM to about 100 mM, about 70 mM to about 90 mM, about 70 mM to about 80 mM, about 80 mM to about 100 mM, about 80 mM to about 90 mM, or about 90 mM to about 100 mM. In some embodiments, the composition comprises Tris (tris(hydroxymethyl) aminomethane or 2-amino-2-(hydroxymethyl) propane-1,3-diol) at a concentration of about 20 mM to 30 mM. In some embodiments, the composition comprises Tris (tris(hydroxymethyl)aminomethane or 2-amino-2-(hydroxymethyl) propane-1,3-diol) at a concentration of 25 mM.

In some embodiments, one of the one or more salts is at a concentration of about 1 mM to 1000 mM, for example a concentration of about 1 mM to about 900 mM, about 1 mM to about 800 mM, about 1 mM to about 700 mM, about 1 mM to about 600 mM, about 1 mM to about 500 mM, about 1 mM to about 400 mM, about 1 mM to about 300 mM, about 1 mM to about 200 mM, about 1 mM to about 100 mM, about 10 mM to about 1000 mM, about 10 mM to about 900 mM, about 10 mM to about 800 mM, about 10 mM to about 700 mM, about 10 mM to about 600 mM, about 10 mM to about 500 mM, about 10 mM to about 400 mM, about 10 mM to about 300 mM, about 10 mM to about 200 mM, about 10 mM to about 100 mM, about 50 mM to about 1000 mM, about 50 mM to about 900 mM, about 50 mM to about 800 mM, about 50 mM to about 700 mM, about 50 mM to about 600 mM, about 50 mM to about 500 mM, about 50 mM to about 400 mM, about 50 mM to about 300 mM, about 50 mM to about 200 mM, about 50 mM to about 100 mM, about 100 mM to about 1000 mM, about 100 mM to about 900 mM, about 100 mM to about 800 mM, about 100 mM to about 700 mM, about 100 mM to about 600 mM, about 100 mM to about 500 mM, about 100 mM to about 400 mM, about 100 mM to about 300 mM or about 100 mM to about 200 mM.

In some embodiments, the composition comprises sodium chloride (NaCl) at a concentration of about 1 mM to 1000 mM, for example a concentration of about 1 mM to about 900 mM, about 1 mM to about 800 mM, about 1 mM to about 700 mM, about 1 mM to about 600 mM, about 1 mM to about 500 mM, about 1 mM to about 400 mM, about 1 mM to about 300 mM, about 1 mM to about 200 mM, about 1 mM to about 100 mM, about 10 mM to about 1000 mM, about 10 mM to about 900 mM, about 10 mM to about 800 mM, about 10 mM to about 700 mM, about 10 mM to about 600 mM, about 10 mM to about 500 mM, about 10 mM to about 400 mM, about 10 mM to about 300 mM, about 10 mM to about 200 mM, about 10 mM to about 100 mM, about 50 mM to about 1000 mM, about 50 mM to about 900 mM, about 50 mM to about 800 mM, about 50 mM to about 700 mM, about 50 mM to about 600 mM, about 50 mM to about 500 mM, about 50 mM to about 400 mM, about 50 mM to about 300 mM, about 50 mM to about 200 mM, about 50 mM to about 100 mM, about 100 mM to about 1000 mM, about 100 mM to about 900 mM, about 100 mM to about 800 mM, about 100 mM to about 700 mM, about 100 mM to about 600 mM, about 100 mM to about 500 mM, about 100 mM to about 400 mM, about 100 mM to about 300 mM or about 100 mM to about 200 mM. In some embodiments, the composition comprises sodium chloride (NaCl) at a concentration of about 200 mM to about 400 mM. In some embodiments, the composition comprises sodium chloride (NaCl) at a concentration of about 300 mM to about 500 mM. In some embodiments, the composition comprises sodium chloride (NaCl) at a concentration of about 100 mM to about 200 mM. In some embodiments, the composition comprises sodium chloride (NaCl) at a concentration of 400 mM. In some embodiments, the composition comprises sodium chloride (NaCl) at a concentration of 275 mM. In some embodiments, the composition comprises sodium chloride (NaCl) at a concentration of 150 mM.

In some embodiments, one of the one or more salts is at a concentration of about 1 mM to about 200 mM, for example a concentration of about 1 mM to about 150 mM, about 1 mM to about 100 mM, about 1 mM to about 50 mM, about 1 mM to about 40 mM, about 1 mM to about 30 mM, about 1 mM to about 20 mM, about 1 mM to about 10 mM, about 5 mM to about 200 mM, about 5 mM to about 150 mM, about 5 mM to about 100 mM, about 5 mM to about 50 mM, about 5 mM to about 40 mM, about 5 mM to about 30 mM, about 5 mM to about 20 mM, about 5 mM to about 10 mM, about 10 mM to about 200 mM, about 10 mM to about 150 mM, about 10 mM to about 100 mM, about 10 mM to about 50 mM, about 10 mM to about 40 mM, about 10 mM to about 30 mM, about 10 mM to about 20 mM, about 15 mM to about 200 mM, about 15 mM to about 150 mM, about 15 mM to about 100 mM, about 15 mM to about 50 mM, about 15 mM to about 40 mM, about 15 mM to about 30 mM or about 15 mM to about 20 mM.

In some embodiments, the composition comprises calcium chloride (CaCl2) at a concentration of about 1 mM to about 200 mM, for example a concentration of about 1 mM to about 150 mM, about 1 mM to about 100 mM, about 1 mM to about 50 mM, about 1 mM to about 40 mM, about 1 mM to about 30 mM, about 1 mM to about 20 mM, about 1 mM to about 10 mM, about 5 mM to about 200 mM, about 5 mM to about 150 mM, about 5 mM to about 100 mM, about 5 mM to about 50 mM, about 5 mM to about 40 mM, about 5 mM to about 30 mM, about 5 mM to about 20 mM, about 5 mM to about 10 mM, about 10 mM to about 200 mM, about 10 mM to about 150 mM, about 10 mM to about 100 mM, about 10 mM to about 50 mM, about 10 mM to about 40 mM, about 10 mM to about 30 mM, about 10 mM to about 20 mM, about 15 mM to about 200 mM, about 15 mM to about 150 mM, about 15 mM to about 100 mM, about 15 mM to about 50 mM, about 15 mM to about 40 mM, about 15 mM to about 30 mM or about 15 mM to about 20 mM.

In some embodiments, the composition comprises calcium chloride (CaCl₂)) at a concentration of about 5 mM to 20 mM. In some embodiments, the composition comprises calcium chloride (CaCl₂)) at a concentration of about 10 mM to 20 mM. In some embodiments, the composition comprises calcium chloride (CaCl₂)) at a concentration of 16 mM. In some embodiments, the composition comprises calcium chloride (CaCl₂)) at a concentration of 8 mM. In some embodiments, the composition comprises tris-buffered saline (TBS).

In some embodiments, the composition comprises Tris (tris(hydroxymethyl) aminomethane or 2-amino-2-(hydroxymethyl) propane-1,3-diol) at a concentration of about 20 mM to 30 mM and comprises NaCl at a concentration of about 100 mM to 200 mM. In some embodiments, the composition comprises Tris (tris(hydroxymethyl) aminomethane or 2-amino-2-(hydroxymethyl) propane-1,3-diol) at a concentration of 25 mM and comprises NaCl at a concentration of 150 mM. In some embodiments, the composition comprises Tris (tris(hydroxymethyl) aminomethane or 2-amino-2-(hydroxymethyl) propane-1,3-diol) at a concentration of about 20 mM to about 30 mM and comprises NaCl at a concentration of about 200 mM to 400 mM. In some embodiments, the composition comprises Tris (tris(hydroxymethyl) aminomethane or 2-amino-2-(hydroxymethyl) propane-1,3-diol) at a concentration of about 25 mM and comprises NaCl at a concentration of about 275 mM.

In some embodiments, the composition comprises Tris (tris(hydroxymethyl) aminomethane or 2-amino-2-(hydroxymethyl) propane-1,3-diol) at a concentration of about 20 mM to about 30 mM, NaCl at a concentration of about 200 mM to about 400 mM and CaCl₂) at a concentration of about 5 mM to about 20 mM. In some embodiments the composition comprises Tris (tris(hydroxymethyl) aminomethane or 2-amino-2-(hydroxymethyl) propane-1,3-diol) at a concentration of about 25 mM, NaCl at a concentration of about 275 mM and CaCl₂) at a concentration of about 8 mM.

In some embodiments, the composition comprises Tris (tris(hydroxymethyl) aminomethane or 2-amino-2-(hydroxymethyl) propane-1,3-diol) at a concentration of about 20 mM to 30 mM, NaCl at a concentration of about 300 mM to 500 mM and CaCl₂) at a concentration of about 10 mM to about 20 mM. In some embodiments, the composition comprises Tris (tris(hydroxymethyl) aminomethane or 2-amino-2-(hydroxymethyl) propane-1,3-diol) at a concentration of about 25 mM, NaCl at a concentration of about 400 mM and CaCl₂) at a concentration of about 16 mM.

In some embodiments, the composition comprises one or more block copolymers at a total concentration of about 0.05% (w/v) to about 5% (w/v), for example about 0.05% (w/v) to about 4.5% (w/v), about 0.05% (w/v) to about 4% (w/v), about 0.05% (w/v) to about 3.5% (w/v), about 0.05% (w/v) to about 3% (w/v), about 0.05% (w/v) to about 2.5% (w/v), about 0.05% (w/v) to about 2% (w/v), about 0.05% (w/v) to about 1.5% (w/v), about 0.05% (w/v) to about 1% (w/v), about 0.05% (w/v) to about 0.5% (w/v), about 0.1% (w/v) to about 5% (w/v), about 0.1% (w/v) to about 4.5% (w/v), about 0.1% (w/v) to about 4% (w/v), about 0.1% (w/v) to about 3.5% (w/v), about 0.1% (w/v) to about 3% (w/v), about 0.1% (w/v) to about 2.5% (w/v), about 0.1% (w/v) to about 2% (w/v), about 0.1% (w/v) to about 1.5% (w/v), about 0.1% (w/v) to about 1% (w/v), about 0.1% (w/v) to about 0.5% (w/v), about 0.5% (w/v) to about 5% (w/v), about 0.5% (w/v) to about 4.5% (w/v), about 0.5% (w/v) to about 4% (w/v), about 0.5% (w/v) to about 3.5% (w/v), about 0.5% (w/v) to about 3% (w/v), about 0.5% (w/v) to about 2.5% (w/v), about 0.5% (w/v) to about 2% (w/v), about 0.5% (w/v) to about 1.5% (w/v), about 0.5% (w/v) to about 1% (w/v), about 0.75% (w/v) to about 5% (w/v), about 0.75% (w/v) to about 4.5% (w/v), about 0.75% (w/v) to about 4% (w/v), about 0.75% (w/v) to about 3.5% (w/v), about 0.75% (w/v) to about 3% (w/v), about 0.75% (w/v) to about 2.5% (w/v), about 0.75% (w/v) to about 2% (w/v), about 0.75% (w/v) to about 1.5% (w/v) or about 0.75% (w/v) to about 1% (w/v). In some embodiments, the composition comprises one or more block copolymers at a total concentration of about 0.75% (w/v) to 1.5% (w/v). In some embodiments, the composition comprises one or more block copolymers at a total concentration of 1% (w/v). In some embodiments, the composition comprises one block copolymer at a concentration of 1% (w/v).

In some embodiments, the composition comprises one or more block copolymers at a total concentration of about 0.05% (w/v) to 0.5% (w/v). In some embodiments, the composition comprises one or more block copolymers at a total concentration of 0.25% (w/v). In some embodiments, the composition comprises one block copolymer at a concentration of 0.25% (w/v). In some embodiments, the composition comprises one or more block copolymers at a total concentration of 0.125% (w/v). In some embodiments, the composition comprises one block copolymer at a concentration of 0.125% (w/v).

In some embodiments, the composition comprises one or more blocking agents at a total concentration of about 0.05% (w/v) to about 10% (w/v), for example about 0.05% (w/v) to about 9% (w/v), about 0.05% (w/v) to about 8% (w/v), about 0.05% (w/v) to about 7% (w/v), about 0.05% (w/v) to about 6% (w/v), about 0.05% (w/v) to about 5% (w/v), about 0.05% (w/v) to about 4% (w/v), about 0.05% (w/v) to about 3% (w/v), about 0.05% (w/v) to about 2.5% (w/v), about 0.05% (w/v) to about 2% (w/v), about 0.05% (w/v) to about 1.5% (w/v), about 0.05% (w/v) to about 1% (w/v), about 0.05% (w/v) to about 0.5% (w/v), about 0.05% (w/v) to about 0.4% (w/v), about 0.05% (w/v) to about 0.3% (w/v), about 0.05% (w/v) to about 0.2% (w/v), about 0.05% (w/v) to about 0.1% (w/v), about 0.1% (w/v) to about 10% (w/v), about 0.1% (w/v) to about 9% (w/v), about 0.1% (w/v) to about 8% (w/v), about 0.1% (w/v) to about 7% (w/v), about 0.1% (w/v) to about 6% (w/v), about 0.1% (w/v) to about 5% (w/v), about 0.1% (w/v) to about 4% (w/v), about 0.1% (w/v) to about 3% (w/v), about 0.1% (w/v) to about 2.5% (w/v), about 0.1% (w/v) to about 2% (w/v), about 0.1% (w/v) to about 1.5% (w/v), about 0.1% (w/v) to about 1% (w/v), about 0.1% (w/v) to about 0.5% (w/v), about 0.1% (w/v) to about 0.4% (w/v), about 0.1% (w/v) to about 0.3% (w/v), about 0.1% (w/v) to about 0.2% (w/v), about 0.2% (w/v) to about 10% (w/v), about 0.2% (w/v) to about 9% (w/v), about 0.2% (w/v) to about 8% (w/v), about 0.2% (w/v) to about 7% (w/v), about 0.2% (w/v) to about 6% (w/v), about 0.2% (w/v) to about 5% (w/v), about 0.2% (w/v) to about 4% (w/v), about 0.2% (w/v) to about 3% (w/v), about 0.2% (w/v) to about 2.5% (w/v), about 0.2% (w/v) to about 2% (w/v), about 0.2% (w/v) to about 1.5% (w/v), about 0.2% (w/v) to about 1% (w/v), about 0.2% (w/v) to about 0.5% (w/v), about 0.2% (w/v) to about 0.4% (w/v), about 0.2% (w/v) to about 0.3% (w/v), about 0.3% (w/v) to about 10% (w/v), about 0.3% (w/v) to about 9% (w/v), about 0.3% (w/v) to about 8% (w/v), about 0.3% (w/v) to about 7% (w/v), about 0.3% (w/v) to about 6% (w/v), about 0.3% (w/v) to about 5% (w/v), about 0.3% (w/v) to about 4% (w/v), about 0.3% (w/v) to about 3% (w/v), about 0.3% (w/v) to about 2.5% (w/v), about 0.3% (w/v) to about 2% (w/v), about 0.3% (w/v) to about 1.5% (w/v), about 0.3% (w/v) to about 1% (w/v), about 0.3% (w/v) to about 0.5% (w/v), about 0.3% (w/v) to about 0.4% (w/v), about 0.4% (w/v) to about 10% (w/v), about 0.4% (w/v) to about 9% (w/v), about 0.4% (w/v) to about 8% (w/v), about 0.4% (w/v) to about 7% (w/v), about 0.4% (w/v) to about 6% (w/v), about 0.4% (w/v) to about 5% (w/v), about 0.4% (w/v) to about 4% (w/v), about 0.4% (w/v) to about 3% (w/v), about 0.4% (w/v) to about 2.5% (w/v), about 0.4% (w/v) to about 2% (w/v), about 0.4% (w/v) to about 1.5% (w/v), about 0.4% (w/v) to about 1% (w/v), about 0.4% (w/v) to about 0.5% (w/v), about 0.5% (w/v) to about 10% (w/v), about 0.5% (w/v) to about 9% (w/v), about 0.5% (w/v) to about 8% (w/v), about 0.5% (w/v) to about 7% (w/v), about 0.5% (w/v) to about 6% (w/v), about 0.5% (w/v) to about 5% (w/v), about 0.5% (w/v) to about 4% (w/v), about 0.5% (w/v) to about 3% (w/v), about 0.5% (w/v) to about 2.5% (w/v), about 0.5% (w/v) to about 2% (w/v), about 0.5% (w/v) to about 1.5% (w/v), about 0.5% (w/v) to about 1% (w/v), about 1% (w/v) to about 10% (w/v), about 1% (w/v) to about 9% (w/v), about 1% (w/v) to about 8% (w/v), about 1% (w/v) to about 7% (w/v), about 1% (w/v) to about 6% (w/v), about 1% (w/v) to about 5% (w/v), about 1% (w/v) to about 4% (w/v), about 1% (w/v) to about 3% (w/v), about 1% (w/v) to about 2.5% (w/v), about 1% (w/v) to about 2% (w/v), about 1% (w/v) to about 1.5% (w/v), about 1.5% (w/v) to about 10% (w/v), about 1.5% (w/v) to about 9% (w/v), about 1.5% (w/v) to about 8% (w/v), about 1.5% (w/v) to about 7% (w/v), about 1.5% (w/v) to about 6% (w/v), about 1.5% (w/v) to about 5% (w/v), about 1.5% (w/v) to about 4% (w/v), about 1.5% (w/v) to about 3% (w/v), about 1.5% (w/v) to about 2.5% (w/v), about 1.5% (w/v) to about 2% (w/v), about 2% (w/v) to about 10% (w/v), about 2% (w/v) to about 9% (w/v), about 2% (w/v) to about 8% (w/v), about 2% (w/v) to about 7% (w/v), about 2% (w/v) to about 6% (w/v), about 2% (w/v) to about 5% (w/v), about 2% (w/v) to about 4% (w/v), about 2% (w/v) to about 3% (w/v), about 2% (w/v) to about 2.5% (w/v), about 2.5% (w/v) to about 10% (w/v), about 2.5% (w/v) to about 9% (w/v), about 2.5% (w/v) to about 8% (w/v), about 2.5% (w/v) to about 7% (w/v), about 2.5% (w/v) to about 6% (w/v), about 2.5% (w/v) to about 5% (w/v), about 2.5% (w/v) to about 4% (w/v), about 2.5% (w/v) to about 3% (w/v), about 3% (w/v) to about 10% (w/v), about 3% (w/v) to about 9% (w/v), about 3% (w/v) to about 8% (w/v), about 3% (w/v) to about 7% (w/v), about 3% (w/v) to about 6% (w/v), about 3% (w/v) to about 5% (w/v), about 3% (w/v) to about 4% (w/v), about 4% (w/v) to about 10% (w/v), about 4% (w/v) to about 9% (w/v), about 4% (w/v) to about 8% (w/v), about 4% (w/v) to about 7% (w/v), about 4% (w/v) to about 6% (w/v), about 4% (w/v) to about 5% (w/v), about 5% (w/v) to about 10% (w/v), about 5% (w/v) to about 9% (w/v), about 5% (w/v) to about 8% (w/v), about 5% (w/v) to about 7% (w/v), about 5% (w/v) to about 6% (w/v), about 6% (w/v) to about 10% (w/v), about 6% (w/v) to about 9% (w/v), about 6% (w/v) to about 8% (w/v), about 6% (w/v) to about 7% (w/v), about 7% (w/v) to about 10% (w/v), about 7% (w/v) to about 9% (w/v), about 7% (w/v) to about 8% (w/v), about 8% (w/v) to about 10% (w/v), about 8% (w/v) to about 9% (w/v) or about 9% (w/v) to about 10% (w/v).

In some embodiments, the composition comprises a blocking agent comprising bovine serum albumin at a concentration of about 0.05% (w/v) to about 10% (w/v). In some embodiments, the composition comprises a blocking agent comprising bovine serum albumin at a concentration of about 0.5% (w/v). In some embodiments, the composition comprises a blocking agent comprising bovine serum albumin at a concentration of about 5% (w/v). In some embodiments, the composition comprises a blocking agent comprising bovine serum albumin at a concentration of about 2.5% (w/v).

In some embodiments, the composition comprises Tris (tris(hydroxymethyl) aminomethane or 2-amino-2-(hydroxymethyl) propane-1,3-diol) at a concentration of about 1 mM to about 500 mM, sodium chloride (NaCl) at a concentration of about 1 mM to about 1000 mM, calcium chloride (CaCl₂)) at a concentration of about 1 mM to 200 mM, bovine serum albumin (BSA) at a concentration of about 0.05% (w/v) to about 10% (w/v) and one or more block copolymers at a total concentration of about 0.05% (w/v) to 5% (w/v). In some embodiments, the composition comprises Tris (tris(hydroxymethyl) aminomethane or 2-amino-2-(hydroxymethyl) propane-1,3-diol) at a concentration of 25 mM, sodium chloride (NaCl) at a concentration of 400 mM, calcium chloride (CaCl₂)) at a concentration of 16 mM, bovine serum albumin (BSA) at a concentration of 5% (w/v) and a polyoxyethylene-polyoxypropylene block copolymer (such as Synperonic® F 108) at a concentration of 0.25% (w/v). In some embodiments, the composition comprises Tris (tris(hydroxymethyl) aminomethane or 2-amino-2-(hydroxymethyl) propane-1,3-diol) at a concentration of 25 mM, sodium chloride (NaCl) at a concentration of 275 mM, calcium chloride (CaCl₂)) at a concentration of 8 mM, bovine serum albumin (BSA) at a concentration of 2.5% (w/v) and a polyoxyethylene-polyoxypropylene block copolymer (such as Synperonic® F 108) at a concentration of about 0.125% (w/v).

In some embodiments, the composition comprises casein at a concentration of about 0.1% (w/v) to about 3% (w/v), for example about 0.1% (w/v) to about 2.5% (w/v), about 0.1% (w/v) to about 2% (w/v), about 0.1% (w/v) to about 1.5% (w/v), about 0.1% (w/v) to about 1% (w/v), about 0.1% (w/v) to about 0.5% (w/v), about 0.1% (w/v) to about 0.4% (w/v), about 0.1% (w/v) to about 0.3% (w/v), about 0.1% (w/v) to about 0.2% (w/v), about 0.2% (w/v) to about 3% (w/v), about 0.2% (w/v) to about 2.5% (w/v), about 0.2% (w/v) to about 2% (w/v), about 0.2% (w/v) to about 1.5% (w/v), about 0.2% (w/v) to about 1% (w/v), about 0.2% (w/v) to about 0.5% (w/v), about 0.2% (w/v) to about 0.4% (w/v), about 0.2% (w/v) to about 0.3% (w/v), about 0.3% (w/v) to about 3% (w/v), about 0.3% (w/v) to about 2.5% (w/v), about 0.3% (w/v) to about 2% (w/v), about 0.3% (w/v) to about 1.5% (w/v), about 0.3% (w/v) to about 1% (w/v), about 0.3% (w/v) to about 0.5% (w/v), about 0.3% (w/v) to about 0.4% (w/v), about 0.4% (w/v) to about 3% (w/v), about 0.4% (w/v) to about 2.5% (w/v), about 0.4% (w/v) to about 2% (w/v), about 0.4% (w/v) to about 1.5% (w/v), about 0.4% (w/v) to about 1% (w/v), about 0.4% (w/v) to about 0.5% (w/v), about 0.5% (w/v) to about 3% (w/v), about 0.5% (w/v) to about 2.5% (w/v), about 0.5% (w/v) to about 2% (w/v), about 0.5% (w/v) to about 1.5% (w/v), about 0.5% (w/v) to about 1% (w/v), about 1% (w/v) to about 3% (w/v), about 1% (w/v) to about 2.5% (w/v), about 1% (w/v) to about 2% (w/v), about 1% (w/v) to about 1.5% (w/v), about 1.5% (w/v) to about 3% (w/v), about 1.5% (w/v) to about 2.5% (w/v), about 1.5% (w/v) to about 2% (w/v), about 2% (w/v) to about 3% (w/v), about 2% (w/v) to about 2.5% (w/v) or about 2.5% (w/v) to about 3% (w/v).

In some embodiments, the composition comprises casein at a concentration of about 0.4% (w/v) to 0.6% (w/v). In some embodiments, the composition comprises casein at a concentration of 0.5% (w/v).

In some embodiments, the composition comprises Tris (tris(hydroxymethyl) aminomethane or 2-amino-2-(hydroxymethyl) propane-1,3-diol), sodium chloride (NaCl), calcium chloride (CaCl₂)), casein and one or more block copolymers.

In some embodiments, the composition comprises Tris (tris(hydroxymethyl) aminomethane or 2-amino-2-(hydroxymethyl) propane-1,3-diol) at a concentration of about 1 mM to 500 mM, sodium chloride (NaCl) at a concentration of about 1 mM to 1000 mM, calcium chloride (CaCl₂)) at a concentration of about 1 mM to 200 mM, casein at a concentration of about 0.1% (w/v) to 2% (w/v) and one or more block copolymers at a total concentration of about 0.05% (w/v) to 5% (w/v). In some embodiments, the composition comprises Tris (tris(hydroxymethyl) aminomethane or 2-amino-2-(hydroxymethyl) propane-1,3-diol) at a concentration of 25 mM, sodium chloride (NaCl) at a concentration of 150 mM, calcium chloride (CaCl₂)) at a concentration of 16 mM, casein at a concentration of 0.5% (w/v) and a polyoxyethylene-polyoxypropylene block copolymer (such as Synperonic® F 108) at a concentration of 1% (w/v).

In some embodiments the block copolymer is a polyoxyethylene-polyoxypropylene block copolymer. In some embodiments, the polyoxyethylene-polyoxypropylene block copolymer is represented by Formula I

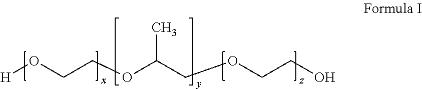

wherein x and z=2-130 and b=15-67. In some embodiments, x and z=120-130 and b=50-60. In some embodiments, the block copolymer is Synperonic® F 108 having the following formula:

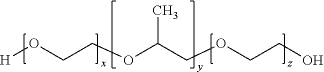

In some embodiments, the composition further comprises one or more chaotropic agents. In some embodiments, the composition further comprises one or more denaturing agents. In some embodiments, the composition further comprises one or more chaotropic and denaturing agents.

In some embodiments, the one or more chaotropic and/or denaturing agents are selected from a list consisting of: a guanidine salt, urea or betaine. In some embodiments, the one or more chaotropic and/or denaturing agents is guanidine hydrochloride.

In some embodiments, the one or more chaotropic and/or denaturing agents are at a concentration of about 1 mM to about 1000 mM, for example a concentration of about 1 mM to about 900 mM, about 1 mM to about 800 mM, about 1 mM to about 700 mM, about 1 mM to about 600 mM, about 1 mM to about 500 mM, about 1 mM to about 400 mM, about 1 mM to about 300 mM, about 1 mM to about 200 mM, about 1 mM to about 100 mM, about 10 mM to about 1000 mM, about 10 mM to about 900 mM, about 10 mM to about 800 mM, about 10 mM to about 700 mM, about 10 mM to about 600 mM, about 10 mM to about 500 mM, about 10 mM to about 400 mM, about 10 mM to about 300 mM, about 10 mM to about 200 mM, about 10 mM to about 100 mM, about 50 mM to about 1000 mM, about 50 mM to about 900 mM, about 50 mM to about 800 mM, about 50 mM to about 700 mM, about 50 mM to about 600 mM, about 50 mM to about 500 mM, about 50 mM to about 400 mM, about 50 mM to about 300 mM, about 50 mM to about 200 mM, about 50 mM to about 100 mM, about 100 mM to about 1000 mM, about 100 mM to about 900 mM, about 100 mM to about 800 mM, about 100 mM to about 700 mM, about 100 mM to about 600 mM, about 100 mM to about 500 mM, about 100 mM to about 400 mM, about 100 mM to about 300 mM or about 100 mM to about 200 mM.

In some embodiments, the composition comprises Tris (tris(hydroxymethyl) aminomethane or 2-amino-2-(hydroxymethyl) propane-1,3-diol) at a concentration of about 1 mM to 500 mM, sodium chloride (NaCl) at a concentration of about 1 mM to 1000 mM, calcium chloride (CaCl₂)) at a concentration of about 1 mM to 200 mM, casein at a concentration of about 0.1% (w/v) to 2% (w/v), one or more block copolymers at a total concentration of about 0.05% (w/v) to 5% (w/v) and one or more chaotropic and/or denaturing agents at a concentration of about 1 mM to about 1000 mM. In some embodiments, the composition comprises Tris (tris(hydroxymethyl) aminomethane or 2-amino-2-(hydroxymethyl) propane-1,3-diol) at a concentration of 25 mM, sodium chloride (NaCl) at a concentration of 150 mM, calcium chloride (CaCl₂)) at a concentration of 16 mM, casein at a concentration of 0.5% (w/v), a polyoxyethylene-polyoxypropylene block copolymer (such as Synperonic® F 108) at a concentration of 1% (w/v) and guanidine hydrochloride (guanidine HCl) at a concentration of 200 mM.

In some embodiments, the composition comprises Tris (tris(hydroxymethyl) aminomethane or 2-amino-2-(hydroxymethyl) propane-1,3-diol) at a concentration of about 1 mM to about 500 mM, sodium chloride (NaCl) at a concentration of about 1 mM to about 1000 mM, calcium chloride (CaCl₂)) at a concentration of about 1 mM to about 200 mM, bovine serum albumin at a concentration of about 0.5% (w/v) to about 10% (w/v), one or more block copolymers at a total concentration of about 0.05% (w/v) to about 5% (w/v) and one or more chaotropic and/or denaturing agents at a concentration of about 1 mM to about 1000 mM. In embodiments, the composition comprises Tris some (tris(hydroxymethyl) aminomethane or 2-amino-2-(hydroxymethyl) propane-1,3-diol) at a concentration of about 25 mM, sodium chloride (NaCl) at a concentration of about 275 mM, calcium chloride (CaCl₂)) at a concentration of about 8 mM, bovine serum albumin at a concentration of about 2.5% (w/v), a polyoxyethylene-polyoxypropylene block copolymer (such as Synperonic® F 108) at a concentration of about 0.125% (w/v) and guanidine hydrochloride (guanidine HCl) at a concentration of about 300 mM.

The present invention also provides a composition for immunoassays comprising one or more chaotropic and/or denaturing agents. In some embodiments, the one or more chaotropic and/or denaturing agents are selected from a list consisting of: a guanidine salt, urea or betaine. In some embodiments, the one or more chaotropic and/or denaturing agents is guanidine hydrochloride.

In some embodiments, the one or more chaotropic and/or denaturing agents are at a concentration of about 1 mM to about 1000 mM, for example a concentration of about 1 mM to about 900 mM, about 1 mM to about 800 mM, about 1 mM to about 700 mM, about 1 mM to about 600 mM, about 1 mM to about 500 mM, about 1 mM to about 400 mM, about 1 mM to about 300 mM, about 1 mM to about 200 mM, about 1 mM to about 100 mM, about 10 mM to about 1000 mM, about 10 mM to about 900 mM, about 10 mM to about 800 mM, about 10 mM to about 700 mM, about 10 mM to about 600 mM, about 10 mM to about 500 mM, about 10 mM to about 400 mM, about 10 mM to about 300 mM, about 10 mM to about 200 mM, about 10 mM to about 100 mM, about 50 mM to about 1000 mM, about 50 mM to about 900 mM, about 50 mM to about 800 mM, about 50 mM to about 700 mM, about 50 mM to about 600 mM, about 50 mM to about 500 mM, about 50 mM to about 400 mM, about 50 mM to about 300 mM, about 50 mM to about 200 mM, about 50 mM to about 100 mM, about 100 mM to about 1000 mM, about 100 mM to about 900 mM, about 100 mM to about 800 mM, about 100 mM to about 700 mM, about 100 mM to about 600 mM, about 100 mM to about 500 mM, about 100 mM to about 400 mM, about 100 mM to about 300 mM or about 100 mM to about 200 mM.

In some embodiments, the composition further comprises a proteinaceous matrix, optionally wherein the proteinaceous matrix comprises bovine serum albumin (BSA) and/or casein.

In some embodiments, the composition further comprises a biocide, optionally wherein the biocide comprises 2-methyl-4-isothiazolin-3-one (MIT), 5-chloro-2-methyl-4-isothiazolin-3-one (CMIT), or a mixture thereof (for example a ProClin™ biocide, such as ProClin™ 300).

In some embodiments, the composition comprises the biocide at a concentration of about 0.01% (w/v) to about 2% (w/v), for example about 0.01% (w/v) to about 1% (w/v), about 0.01% (w/v) to about 0.5% (w/v), about 0.01% (w/v) to about 0.1% (w/v), about 0.02% (w/v) to about 0.08% (w/v), about 0.03% (w/v) to about 0.07% (w/v), or about 0.05% (w/v).

In some embodiments, the composition further comprises an anti-foaming agent, optionally wherein the anti-foaming agent comprises non-silicone defoamers in a polyol-based dispersion (for example Antifoam 204).

In some embodiments, the composition comprises the anti-foaming agent at a concentration of about 0.01% (w/v) to about 2% (w/v), for example about 0.01% (w/v) to about 1% (w/v), about 0.01% (w/v) to about 0.5% (w/v), about 0.01% (w/v) to about 0.1% (w/v), about 0.02% (w/v) to about 0.08% (w/v), about 0.03% (w/v) to about 0.07% (w/v), or about 0.05% (w/v).

In some embodiments, the composition further comprises a heterophile blocking composition (for example MAB33 lgG1/lgG1 Poly). Heterophile blocking compositions are commonly used in immunoassays to block heterophilic antibody interference.

In some embodiments, the composition is a liquid, a gel or an emulsion. In some embodiments, the composition comprises water. In some embodiments, the composition is lyophilised. A lyophilised composition may comprise components at ratios suitable to obtain the concentrations described herein on addition of liquid, for example water.

In some embodiments, the composition is an assay buffer composition. In some embodiments, the composition is an assay diluent composition. In some embodiments, the composition is an assay washing composition. In some embodiments, the assay diluent composition is an analyte capture diluent. In some embodiments, the assay diluent is an analyte detection diluent. An analyte capture diluent may be used in an immunoassay as a diluent during a step comprising contacting analyte with a capture antibody. An analyte detection diluent may be used in an immunoassay as a diluent during a step comprising contacting analyte with a detection antibody.

In some embodiments, the liquid composition comprises the components of the composition in an aqueous solution, suspension, or combination thereof. Accordingly, the composition may comprise water with the remaining components dissolved or suspended in water. In some embodiments, the composition may be formed by solubilising one or more components of the composition provided in lyophilised form.

The present invention also provides a method for preparing a composition for immunoassays as described herein, wherein the method comprises mixing water with additional components of the composition to provide the composition. The present invention also provides a method for preparing a composition for immunoassays as described herein, wherein the method comprises mixing aqueous buffer solution with additional components of the composition to provide the composition. The additional components may be provided in solid form, in aqueous solution, or a combination thereof. One or more of the additional components may be provided in lyophilised form prior to mixing with the aqueous buffer solution.

The present invention also provides a method for preparing a composition for immunoassays as described herein, wherein the composition comprises a blocking agent, the method comprising: providing a liquid composition comprising a buffer compound, a salt, one or more block copolymers and optionally one or more additional components of the composition; providing lyophilised blocking agent; and mixing the liquid composition with the lyophilised blocking agent to provide the composition for immunoassays.

In some embodiments, wherein the composition further comprises one or more chaotropic and/or denaturing agents, the method further comprises providing the one or more chaotropic and/or denaturing agents in lyophilised form and mixing the liquid composition with the lyophilised blocking agent and the lyophilised one or more chaotropic and/or denaturing agents to provide the composition for immunoassays.

In a method for preparing a composition for immunoassays as described herein, the components of the composition may provided so as to prepare a composition with the components and/or concentrations of any of the embodiments of a composition as described herein. For example, in some embodiments the blocking agent is BSA and/or the one or more chaotropic and/or denaturing agents is a guanidine salt, for example guanidine hydrochloride.

The present invention also provides a method of carrying out an immunoassay, the method comprising contacting a biological sample with one or more compositions according to the invention. In some embodiments of the method, the immunoassay is for detecting presence of an analyte of interest in the biological sample. In some embodiments of the method, the biological sample is contacted with the one or more compositions according to the invention simultaneously or sequentially.

In some embodiments of the method, the one or more compositions is used as a diluent for the biological sample.

In some embodiments of the method, the method comprises contacting the biological sample with a capture antibody, wherein the one or more compositions is used as a capture diluent when the biological sample is contacted with the capture antibody. In some embodiments of the method, the method comprises addition of a detection antibody and the one or more compositions is used as a detection diluent when the detection antibody is added. In some embodiments of the method, addition of the detection antibody may occur simultaneously with or subsequently to contacting of the biological sample with the capture antibody. In some embodiments of the method, a wash step using a washing solution occurs after contacting the biological sample with a capture antibody and the detection diluent is added to the capture antibody prior to or simultaneously with addition of the detection antibody.

In some embodiments of the method, the one or more compositions is used as a washing solution. In some embodiments of the method, the washing solution comprises or consists essentially of tris buffered saline and a block copolymer, as defined in any of the embodiments described herein mutatis mutandis.

In some embodiments of the method, the one or more compositions is used as a buffer.

In some embodiments of the method, the biological sample is a blood sample, for example a whole blood sample or a blood fraction, amniotic fluid; aqueous humour; bile; blood plasma; breast milk; cerebrospinal fluid (CSF), endolymph, extracellular fluid, exudate, gastric acid, haemolymph, interstitial fluid, lymph, mucus, pericardial fluid, peritoneal fluid, perspiration (sweat), phlegm, pus, saliva, semen, synovial fluid, tears, urine, vaginal fluids, vomit, sputum, other biofluid or swab sample. In some embodiments of the method, the biological sample is a whole blood sample.

In some embodiments of the method, the biological sample comprises an analyte of interest, optionally wherein the analyte of interest is a protein. In some embodiments of the method, the analyte of interest is brain natriuretic peptide or N-terminal pro-BNP. In some embodiments of the method, the analyte of interest is cardiac troponin or cardiac troponin subunit I (cTnl).

In some embodiments of the method, the immunoassay is a competitive sandwich immunoassay. In some embodiments of the method, the immunoassay comprises obtaining an electrochemical measurement to detect an analyte of interest. In some embodiments of the method, the electrochemical measurement is an amperometric, voltametric, potentiometric, impedimetric, or electrochemical impedance spectroscopic measurement. In some embodiments of the method, the electrochemical measurement is a chronoamperometric measurement.

In some embodiments of the method, the method is for measuring an analyte of interest in a biological sample and comprises:
a. combining said biological sample with:
   a first antibody or antigen binding portion thereof capable of binding said analyte of interest; a second antibody or antigen binding portion thereof capable of binding said analyte of interest conjugated to an enzyme; and
   an assay diluent, wherein the assay diluent is any composition according to the invention; to form an assay mixture;
b. contacting the assay mixture with a substrate for the enzyme, wherein the enzyme substrate is converted by the enzyme into an electroactive molecule; and
C. obtaining an electrochemical measurement to measure the analyte of interest.

In some embodiments of the method, the method is for measuring an analyte of interest in a biological sample and comprises:
a. combining said biological sample with:
   magnetically susceptible beads conjugated to a first antibody or antigen binding portion thereof capable of binding said analyte of interest;
   a second antibody or antigen binding portion thereof capable of binding said analyte of interest conjugated to an enzyme; and
   an assay diluent, wherein the assay diluent is any composition according to the invention; to form an assay mixture
b. retaining the magnetically susceptible beads on an electrode using a magnetic field;
c. contacting the magnetically susceptible beads with a substrate for the enzyme, wherein the enzyme substrate is converted by the enzyme into an electroactive molecule; and
d. obtaining an electrochemical measurement using said electrode.

In some embodiments of the method, the method is for measuring an analyte of interest in a biological sample and comprises:
a. combining said biological sample with magnetically susceptible beads conjugated to a first antibody or antigen binding portion thereof capable of binding said analyte of interest in the presence of a capture diluent, wherein the capture diluent is any composition according to the invention;
b. immobilizing the magnetically susceptible beads on a magnet and washing the beads;
c. providing a second antibody or antigen binding portion thereof capable of binding said analyte of interest conjugated to an enzyme in a detection diluent, wherein the detection diluent is any composition according to the invention, to produce a detection solution;
d. adding the detection solution to the washed magnetically susceptible beads;
e. washing the magnetically susceptible beads;
f. contacting the magnetically susceptible beads with a substrate for the enzyme, wherein the enzyme substrate is converted by the enzyme into an electroactive molecule; and
g. obtaining an electrochemical measurement to measure the analyte of interest.

In some embodiments of the method, washing in steps b. and e. is carried out with a washing solution, optionally wherein the washing solution is any composition according to the invention. In some embodiments of the method, the washing solution comprises or consists essentially of tris buffered saline and a block copolymer, as described herein.

In some embodiments of the method, the capture diluent is any composition as described herein, comprising a buffer compound, at least one salt, one or more block copolymers, one or more blocking agents and one or more chaotropic and/or denaturing agents. In some embodiments of the method, the capture diluent comprises at least two salts. In some embodiments of the method, the capture diluent comprises tris, NaCl, CaCl₂), a polyoxyethylene-polyoxypropylene block copolymer (such as Synperonic® F 108), bovine serum albumin and guanidine hydrochloride (guanidine HCl).

In some embodiments of the method, the detection diluent is any composition according to the invention, comprising a buffer compound, at least one salt, one or more block copolymers and one or more blocking agents. In some embodiments of the method, the detection diluent comprises at least two salts. In some embodiments of the method, the detection diluent comprises tris, NaCl, CaCl₂), a polyoxyethylene-polyoxypropylene block copolymer (such as Synperonic® F 108) and bovine serum albumin.

In some embodiments of the method, the one or more compositions according to the invention are prepared by any appropriate method as described herein.

In some embodiments of the method, the immunoassay is performed on a microfluidic cartridge, optionally a microfluidic disposable cartridge. In some embodiments of the method, the one or more compositions according to the invention are prepared by any appropriate method as described herein on the microfluidic cartridge. In some embodiments of the method, the one or more compositions prepared by a method as described herein on the microfluidic cartridge are a capture diluent and/or a detection diluent.

The present invention also provides use of a composition according to the invention in an immunoassay. The present invention also provides use of a composition according to the invention in a point of care diagnostic assay, such as an immunoassay. In some embodiments, the immunoassay is a competitive sandwich immunoassay. In some embodiments, the immunoassay comprises obtaining an electrochemical measurement to detect an analyte of interest. In some embodiments, the electrochemical measurement is an amperometric, voltametric, potentiometric, impedimetric, or electrochemical impedance spectroscopic measurement. In some embodiments, the electrochemical measurement is a chronoamperometric measurement.

The present invention also provides a kit comprising:
one or more compositions according to the invention; and
a point-of-care immunoassay diagnostic test.

In some embodiments the kit further comprises instructional material, optionally wherein the instructional material comprises instructions for conducting a method of carrying out an immunoassay according to the invention.

Any of the embodiments of a composition, method, use or kit of the present invention may be present in combination with any other embodiment mutatis mutandis. For example, the concentrations recited for each of the various components of the composition may be present in combination with any of the concentrations for any of the other components of the concentration. The present disclosure describes compositions of the invention suitable for use in immunoassays. The present disclosure also represents disclosure of any composition described herein per se.

### BRIEF DESCRIPTION OF THE DRAWINGS

Embodiments of the present invention will now be described, by way of example only, with reference to the accompanying drawings, in which:

FIG. 1—Comparison of chronoamperometry measurements from human whole blood samples obtained in a multiwell plate device. Several surfactants were varied in the formulation, with the surfactant identity shown in the figure legend.

FIG. 2—Comparison of chronoamperometry measurements from human whole blood samples obtained in a microfluidic disposable cartridge. Several surfactants were varied in the formulation, with the surfactant identity shown in the figure legend.

FIG. 3—Calibration of immunoassay response to analyte in human whole blood samples. Two surfactants were varied in the formulation of the diluent buffer and the wash buffer, with the surfactant identity shown in the figure legend. The diluent buffer was spiked with various concentrations of analyte from 0 to 200 pg/mL. For each spiked concentration the current signal was estimated at 45 seconds. A linear fit to the concentration response is presented for two formulations.

FIG. 4—Sensitivity of immunoassay response to analyte in human whole blood samples. Two surfactants were varied in the formulation of the diluent buffer and the wash buffer, with the surfactant identity shown in the figure legend. The diluent buffer was spiked with various concentrations of analyte from 0 to 200 pg/mL. For each time point in the chronoamperometry trace a linear model was used to estimate the slope of the concentration response. In all cases the Synperonic® formulation showed a greater sensitivity compared to the Tween formulation.

FIG. 5—Background of immunoassay response to analyte in human whole blood samples. Two surfactants were varied in the formulation of the diluent buffer and the wash buffer, with the surfactant identity shown in the figure legend. The diluent buffer was spiked with various concentrations of analyte from 0 to 200 pg/mL. For each time point in the chronoamperometry trace a linear model was used to estimate the intercept of the concentration response. The Synperonic® formulation shows a relatively flat background signal whereas for the Tween formulation the background spikes considerably after 20 seconds.

FIG. 6—Synperonic® titration in diluent buffer containing bovine serum albumin (BSA) showing increasing assay sensitivity with increasing Synperonic® concentration. Increasing Synperonic® concentration in the sample diluent increases the difference in assay response (chronoamperometry current after 60 seconds) between samples containing zero and 200 ng/L analyte.

FIG. 7—Synperonic® titration in diluent buffer containing casein (BSA) showing increasing assay sensitivity with increasing Synperonic® block copolymer concentration. Increasing Synperonic® concentration in the sample diluent increases the difference in assay response (chronoamperometry current after 60 seconds) between samples containing zero and 200 ng/L analyte.

FIG. 8—Calibration of immunoassay response to analyte using a diluent buffer comprising bovine serum albumin (BSA) and Synperonic® block copolymer in a 96 well plate.

FIG. 9—Calibration of immunoassay response to analyte using a diluent buffer comprising bovine serum albumin (BSA) and Synperonic® block copolymer in a microfluidic disposable cartridge.

FIG. 10—Calibration of immunoassay response to analyte using a diluent buffer comprising casein and Synperonic® block copolymer in a microfluidic disposable cartridge

FIG. 11—Calibration curve for cardiac troponin (cTnl) assay performed on a 96-well plate using diluent formed from liquid and lyophilised components

### DETAILED DESCRIPTION OF THE INVENTION

The above-described methods can be performed in combination with each other, and in many different orders or multiple times, as required for a given diagnostic test. One method need not be completed before another method is performed.

### Block Co-Polymers

Block copolymers comprise two or more homopolymer subunits linked by covalent bonds. The union of the homopolymer subunits may require an intermediate non-repeating subunit, known as a junction block. Di-block copolymers have two distinct blocks; tri-block copolymers have three. A block is a portion of a macromolecule, comprising many units, that has at least one feature which is not present in the adjacent portions. One possible sequence of repeat units A and B in a triblock copolymer might be ˜A-A-A-A-A-A-A-B-B-B-B-B-B-B-A-A-A-A-A˜. In some embodiments of the invention, the block copolymer may have 1 or more distinct blocks, such as 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or more distinct blocks. In a preferred embodiment, the block copolymer is a triblock copolymer, comprising three distinct blocks. The number of repeated units of monomer in each block can be any number that allows the block copolymer to retain at least one desired property. In some embodiments of the invention, the number of repeated units of monomer in each block can be 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 200, 300, 400, 500, 1000, 2000, 3000, 4000, 5000, 10000 or more.

Block copolymers are made up of blocks of different polymerized monomers. For example, polystyrene-b-poly(methyl methacrylate) or PS-b-PMMA (where b=block) is usually made by first polymerizing styrene, and then subsequently polymerizing methyl methacrylate (MMA) from the reactive end of the polystyrene chains. Triblocks, tetrablocks, multiblocks, etc. can also be made. Diblock copolymers are made using living polymerization techniques, such as atom transfer free radical polymerization (ATRP), reversible addition fragmentation chain transfer (RAFT), ring-opening metathesis polymerization (ROMP), chain shuttling polymerization and living cationic or living anionic polymerizations.

The “blockiness” of a copolymer is a measure of the adjacency of comonomers vs their statistical distribution. Many or even most synthetic polymers are in fact copolymers, containing about 1-20% of a minority monomer. In such cases, blockiness is undesirable. A block index has been proposed as a quantitative measure of blockiness or deviation from random monomer composition.

In some embodiments, the block copolymer used in the invention is a poloxamer (also known as Pluronics® or Synperonics®). Poloxamers, are a class of synthetic block copolymers which consist of hydrophilic poly(ethylene oxide) (PEO) and hydrophobic poly(propylene oxide) (PPO), arranged in an A-B-A triblock structure, thus giving PEO-PPO-PEO. They can be found either as liquids, pastes or solids. Due to their amphiphilic characteristics (presence of hydrophobic and hydrophilic components), poloxamers possess surfactant properties which allow them to interact with hydrophobic surfaces and biological molecules. Being amphiphilic also results in the ability of the individual block copolymers, known as unimers, to combine and form micelles in aqueous solutions. When the concentration of the block copolymers is below that of the critical micelle concentration (CMC), the unimers remain as molecular solutions in water. However, as the block copolymer concentration is increased above the CMC, the unimers will self-assemble and form micelles, which can take on spherical, rod-shaped or lamellar geometries. Their shapes depend on the length and concentration of the block copolymers (i.e. EO, ethylene oxide and PO, propylene oxide), and the temperature. Micelles usually have a hydrophobic core, in this case the PO chains, and a hydrophilic shell, the EO chains. [1]

A poloxamer may be represented by Formula I

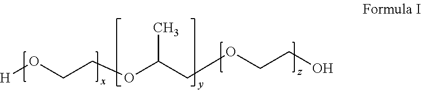

wherein x, y and z are integer values, preferably wherein x and z=2-130 and b=15-67. In some embodiments, x and z may preferably be 120-130 and b may preferably be 50-60. Poloxamers are commonly named with the letter P (for poloxamer) followed by three digits: the first two digits multiplied by 100 give the approximate molecular mass of the polyoxypropylene core, and the last digit multiplied by 10 gives the percentage polyoxyethylene content (e.g. P407=poloxamer with a polyoxypropylene molecular mass of 4000 g/mol and a 70% polyoxyethylene content). The average molecular mass and weight percentage of polyoxyethylene of a poloxamer may be as determined in accordance with the US pharmacopoeia (US Pharmacopeia (2022). NF Monographs, Poloxamer. USP-NF. Rockville, MD; DOI: https://doi.org/10.31003/USPNF_M66210_05_01, incorporated herein by reference). For Pluronics® and Synperonics®, naming of these copolymers starts with a letter to define its physical form at room temperature (L=liquid, P=paste, F=flake (solid)) followed by two or three digits, The first digit (two digits in a three-digit number) in the numerical designation, multiplied by 300, indicates the approximate molecular weight of the hydrophobe; and the last digit×10 gives the percentage polyoxyethylene content.

In some embodiments the block copolymer has a formula according to Formula I

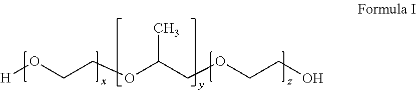

and is poloxamer P338 (e.g. Synperonic® F 108).

Poloxamer P338 may be as defined in the US pharmacopoeia, i.e. having an average molecular mass of 12700 to 17400 and 83.1±1.7 weight % oxyethylene. Synperonic® F 108 indicates a copolymer that is solid (F-Flake) at room temperature and corresponds to a polyoxypropylene molecular mass of 3000 g/mol and a 80% polyoxyethylene content.

### Chaotropic or Denaturing Agents

A chaotropic agent is a substance which disrupts the structure of, and denatures, macromolecules such as proteins and nucleic acids (e.g. DNA and RNA). Chaotropic solutes increase the entropy of the system by interfering with intermolecular interactions mediated by non-covalent forces such as hydrogen bonds, van der Waals forces, and hydrophobic effects.

Macromolecular structure and function is dependent on the net effect of these forces, therefore it follows that an increase in chaotropic solutes in a biological system will denature macromolecules, reduce enzymatic activity and induce stress on a cell.

Tertiary protein folding is dependent on hydrophobic forces from amino acids throughout the sequence of the protein. Chaotropic solutes decrease the net hydrophobic effect of hydrophobic regions because of a disordering of water molecules adjacent to the protein. This solubilises the hydrophobic region in the solution, thereby denaturing the protein. This is also directly applicable to the hydrophobic region in lipid bilayers; if a critical concentration of a chaotropic solute is reached (in the hydrophobic region of the bilayer) then membrane integrity will be compromised, and the cell will lyse.

Chaotropic salts that dissociate in solution exert chaotropic effects via different mechanisms. Whereas chaotropic compounds such as ethanol interfere with non-covalent intramolecular forces as outlined above, salts can have chaotropic properties by shielding charges and preventing the stabilization of salt bridges. Hydrogen bonding is stronger in non-polar media, so salts, which increase the chemical polarity of the solvent, can also destabilize hydrogen bonding. Mechanistically this is because there are insufficient water molecules to effectively solvate the ions. This can result in ion-dipole interactions between the salts and hydrogen bonding species which are more favorable than normal hydrogen bonds.

Examples of chaotropic or denaturing agents suitable for use in compositions of the invention include guanidine salts (such as guanidine hydrochloride), betaine, urea, n-butanol, ethanol, guanidinium chloride, lithium perchlorate, lithium acetate, magnesium chloride, phenol, 2-propanol, sodium dodecyl sulphate and thiourea.

### Biological Samples

In the present invention, the sample liquid may be any suitable biological sample comprising diagnostic biomarkers of interest. In some embodiments, the sample liquid may be a whole blood sample, a serum sample, a saliva sample, a biopsy sample (such as a healthy tissue sample or a tumour sample), a urine sample, a semen sample, a tear sample, a sputum sample, a sweat sample, a mucous sample, a fecal sample, a gastric fluid sample, an abdominal fluid sample, an amniotic fluid sample, a cyst fluid sample, a peritoneal fluid sample, a spinal fluid sample or a synovial fluid sample, although whole blood samples are particularly useful. In a preferred embodiment of the invention the sample liquid is a whole blood sample. The method may include a step of obtaining or providing the biological sample, or alternatively the sample may have already been obtained from a subject, for example in ex vivo methods.

Biological samples obtained from a subject can be stored until needed. Suitable storage methods include freezing immediately, within 2 hours or up to two weeks after sample collection. Maintenance at −80° C. can be used for long-term storage. Preservative may be added, or the sample collected in a tube containing preservative. Preferably the sample is analysed immediately following collection.

Methods of the invention may comprise steps carried out on biological samples. The sample liquid is considered to be representative of the biomarker status of the biomarkers of interest in difference disease states. Hence the methods of the present invention may use quantitative data on biomarkers of interest, to determine the presence, absence or severity of different disease states.

The sample may be processed prior to determining the status of the biomarkers. The sample may be subject to enrichment (for example to increase the concentration of the biomarkers being quantified), centrifugation or dilution. In other embodiments, the samples do not undergo any pre-processing and are used unprocessed (such as whole blood).

In some embodiments of the invention, the biological sample may be fractionated or enriched for particular biomarkers prior to detection and quantification (i.e. measurement). The step of fractionation or enrichment can be any suitable pre-processing method step to increase the concentration of a biomarker of interest in the sample. For example, the steps of fractionation and/or enrichment may comprise centrifugation and/or filtration to remove cells or unwanted analytes from the sample, or to increase the concentration of biomarkers of interest in a particular blood fraction. Such methods may be used to enrich the sample for any biomarkers of interest.

The methods of the invention may be carried out on one test sample from a subject. Alternatively, a plurality of test samples may be taken from a subject, for example at least 2, at least 3, at least 4 or at least 5 samples from a subject. Each sample may be subjected to a single assay to quantify one of the biomarker panel members, or alternatively a sample may be tested for all of the biomarkers being quantified. Each sample may be subjected to a separate analysis using a method of the invention, or alternatively multiple samples from a single subject undergoing diagnosis could be included in the method.

A “sample(s)”, “one or more samples”, sample liquid, or “sample(s) of interest” are terms used interchangeably in singular or plural form and are not intended to be limited to any particular quantity and, as used herein, may be any molecule or substance that the user wishes to gather information from. A sample may become larger or smaller (e.g., by way of inflation or partitioning, respectively) in size, volume or content during the performance of an assay. Accordingly, a sample may be amplified and/or subdivided one or more times during the performance of an assay. In some embodiments, the sample comprises biomarkers of interest.

A “liquid”, as used herein, is any aqueous or lipophilic phase capable of flowing freely. The liquid may further comprise one or more compositions, reaction components or samples of interest selected from cells (including any eukaryotic or prokaryotic cells, including but not limited to cells selected from humans, animals, plants, fungi, bacteria, viruses, protozoa, yeasts, molds, algae, rickettsia, and prions); proteins, peptides, antibodies, nucleic acid sequences, oligonucleotide probes, polymerase enzymes, buffers, dNTPs, organic and inorganic chemicals, and fluorescent dyes.

### Assay Methods

Diagnostic immunoassay methods suitable for use with compositions of the invention may be implemented by a diagnostic system using computer executable instructions. A computer program product or computer readable medium may comprise or store the computer executable instructions. The computer program product or computer readable medium may comprise a hard disk drive, a flash memory, a read-only memory (ROM), a CD, a DVD, a cache, a random-access memory (RAM) and/or any other storage media in which information is stored for any duration (e.g. for extended time periods, permanently, brief instances, for temporarily buffering, and/or for caching of the information). A computer program may comprise the computer executable instructions. The computer readable medium may be a tangible or non-transitory computer readable medium. The term “computer readable” encompasses “machine readable”.

Thus, also disclosed is a computer program comprising computer-executable instructions which, when executed by a diagnostic system, cause the diagnostic system to perform a diagnostic immunoassay method suitable for use with compositions of the invention.

The embodiments are not limited to a microfluidic scale but applications on other, for example macroscopic scales, are equally envisaged. For the avoidance of doubt, the term “microfluidic” is referred to herein to mean devices having a fluidic element such as a reservoir or a channel with at least one dimension below 1 mm.

### Example 1-Use of Block Copolymer Compositions in Stabilizing Electrochemical Signal

Many biosensors that operate in blood use immunoassays as their detection mechanism, often including a sandwich ELISA. The sandwich can terminate with a detection enzyme (e.g. peroxidase enzyme, such as Horseradish Peroxidase-HRP), that is the final component in generating a signal for the sensor.

However, blood components contain haemoglobin, as well as other enzymes such as myeloperoxidases, NADPH oxidases which can be present at the moment of signal generation. These can act as peroxidase mimics, and so their presence can interfere with measurements and lead to false results. Their presence also causes the quality of electrochemical signals to decay, from a smooth, clear response to a highly erratic, noisy response. This is thought to be due to the series of incubation steps, washing steps and changing solutions leading to lysis of blood cells that remain on the assay or sensor, thereby releasing the aforementioned enzymes.

The inventors have discovered that by manipulating the type of surfactant and concentration of surfactant in the diluent and wash solutions, it is possible to alter the role that haemoglobin plays in the electrochemical measurement.

A typical approach in the art is to use Tween-20 for diluents and wash solutions, which leads to unstable erratic electrochemical traces. By changing the diluent to include a block copolymer (such as Synperonic® F108 or pluronics), it is possible to delay the onset of enzymatic interference in the signal, and allow for easier measurement and processing of signal traces, and thus achieve more accurate diagnoses.

The use of block copolymers has significant advantages for the following reasons: (i) The block copolymers are fully synthetic highly stable components in aqueous solutions; (ii) the electrochemical signal is cleaner, smoother and easier to process, overall reducing the error associated with the measurement, (iii) it is possible to measure an electrochemical signal for longer time frames potentially leading to higher assay sensitivity and; (iv) these block copolymers can be introduced to the wash buffer and also act as potent detergents to remove unbound analyte and reduce non-specific signal from the electrode.

Use of a composition according to the invention can also reduce background signal seen with electrochemical detection.

### Example 2-Diluent Buffers Comprising Block Copolymers as Emulsifiers

In most assays, the formulation plays a very important role on the assay's performance. Most standard assays use diluents that contain one or more of the following compositions: a buffer solution, a source of protein, a detergent and additional component to reduce non-specific interactions. The ratio and the selection of the components is critical as it affects many aspects of the assay such as antibody kinetics, sensitivity, precision and reproducibility as well as helping reduce sample to sample variability.

It is desirable that assays are developed to have fast binding kinetics, suitable for point-of-care (PoC) applications as well as low donor to donor variability. In addition, in order for the assay to work in a broad range of samples (e.g. serum, plasma and whole blood), it is required that the formulation can work effectively regardless of the sample matrix and to provide consistent and comparable results.

One such composition includes casein. Casein is widely used as an immunoassay blocker due to its protein bulking effect as well as its low cost. It is a hydrophobic protein that can create micelles in aqueous solutions under certain conditions. The issue is that proteins from the matrix as well as antibodies and enzymes, especially those with large molecular weights, can easily aggregate in such solutions.

Preferred methods that can be implemented with compositions of the invention use enzymes linked to high molecular weight polymers in order to amplify the electrochemical signal. In addition, these polymeric enzyme components are used in high concentrations in order to improve assay kinetics and increase sensitivity. However, these components can generally aggregate in solution, especially when whole blood is used as a matrix.

The solution to this problem is the use of block copolymers to act as emulsifiers. Block copolymers are synthetic molecules that usually contain two hydrophilic and one hydrophobic part. These components can be tailored with different length of monomeric blocks to confer different solubilities in aqueous and hydrophobic environments, which is usually measured by the HLB index Hydrophile-Lipophile Balance.

The use of PEO-PPO-PEO block copolymers to create an emulsion allows for better solubilization of the whole blood matrix as well as the polymeric enzyme components. This has a significant impact in increasing sensitivity of the assay as well as reducing the donor to donor variability.

Additional advantages include the stability of these compositions at room temperature. Furthermore, by varying the molecular weight and the HLB of the polymer it is possible to modify the compositions to work with a variety of assays for multiple biomarkers. In addition, these polymers act as detergents so there is no extra need to add any further detergent to the formulation. Finally, it also acts as a wetting agent.

The combination of a well-known material for immunoassays such as casein with a polymeric material to create an emulsion to improve assay performance has not been disclosed in the prior art, particularly in relation to assays utilising large polymeric recognition molecules for high sensitivity assay applications.

### Example 3-Diluent Buffers Comprising Guanidine Salts

In immunoassays non-specific binding is a very common issue. This can manifest as higher signal than expected and it affects immunoassay sensitivity and limit of detection (LoD) as well as giving erroneous results from unknown concentration samples. Preferred assays that can be implemented with compositions of the invention use high molecular weight polymeric enzyme components to enhance sensitivity and in some cases the assay background electrochemical signal can rise significantly. In addition, clinical samples can also aggregate with antibodies, due to the presence of proteins that bind non-specifically to the antibody antigen complex. This also manifests as higher background non-specific signal which causes assay imprecision.

The use of low concentrations of strong denaturants and chaotropes such as guanidine hydrochloride, urea and betaine, that are traditionally used in molecular biology to denature nucleic acid templates, can also have a beneficial effect in reducing the background signal in immunoassays.

This reduction in background is also accompanied, in some cases, by the reduction in specific signal, but the background reduction is much more favourable. For example, guanidine salts are particularly effective for reducing background in cardiac troponin I assays for at least the following reasons: (i) given the requirement for high sensitivity and low limit of detection, guanidine salts break complexes that cause nonspecific signals, (ii) they markedly reduce aggregation of the large enzyme polymers that can be formed, especially in low analyte concentrations and (iii) they slightly denature the troponin antigen, thereby allowing for better accessibility of antibodies, and reducing the effect of other troponin binding proteins, that in turn can also lead to non-specific interactions. Guanidine salts and similar chaotropes function best when added in a proteinous matrix (BSA, casein).

Other advantages of such compositions include exceptional stability in various conditions, making them suitable components for point of care (PoC) devices where shelf life in various environmental conditions is critical.

### Example 4-Wash Buffers Comprising Block Copolymers

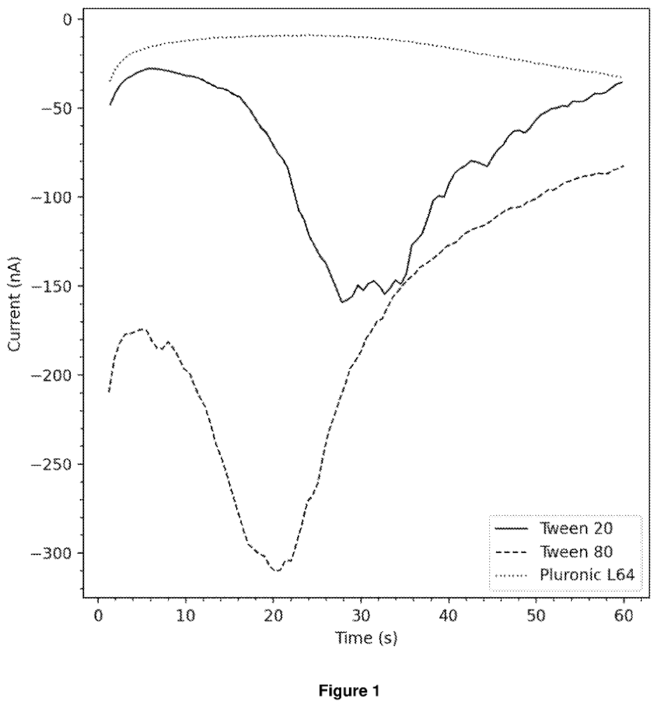

FIG. 1 shows the effect on the quality of the electrochemical signal from adding different surfactants to the wash buffer used to carry out an immunoassay on a 96-well plate. Samples were human whole blood mixed with diluent (25 mL tris buffer, 400 mM NaCl, 16 mM CaCl2, 200 mM guanidine hydrochloride, 1% casein, 0.03% heterophile blocking composition −24), 4 ug/mL detection antibody and magnetic beads coated with capture antibody. This mixture was incubated for several minutes on a 96 well plate at 37° C. This mixture was then emptied and washed twice with 100 uL, each time retaining the magnetic beads. The wash buffer was tris-buffered saline (TBS) mixed with 0.1% of a surfactant, with the surfactant identity shown in the figure legend (see FIG. 1).

After washing the magnetic particles were localised on an 3 mm electrode, and 3,3′,5,5′-tetramethylbenzidine (TMB) solution introduced. A chronoamperometry measurement was undertaken using a three electrode system, with screen-printed carbon used for each of the working, counter and reference electrodes. A voltage of −200 mV was applied for 60s and the current recorded.

TABLE 1
Surfactants investigated. Non-ionic surfactants selected from
Surf's Up ® kit from QED Bioscience Inc.

| | Molecular | |
|---|---|---|
| | Weight | |
| Name | (g/mol) | Application |
| Pluronic ® L64 | 2900 | Non-hemolytic, solvent compatible, |
| Poly(oxyethylene- | | low foaming, good wetter. |
| cooxypropylene) | | |
| block copolymer | | |
| Tween ® 20 | 1228 | Non-hemolytic, very hydrophilic, good |
| Polyoxyethylene | | emulsifier and solubilizer. |
| (20) sorbitan | | |
| monolaurate | | |
| Tween ® 80 | 1310 | Good emulsifier and solubilizer. |
| Polyethylene glycol | | |
| sorbitan | | |
| monooleate | | |

### Example 5-Wash Buffers Comprising Block Copolymers

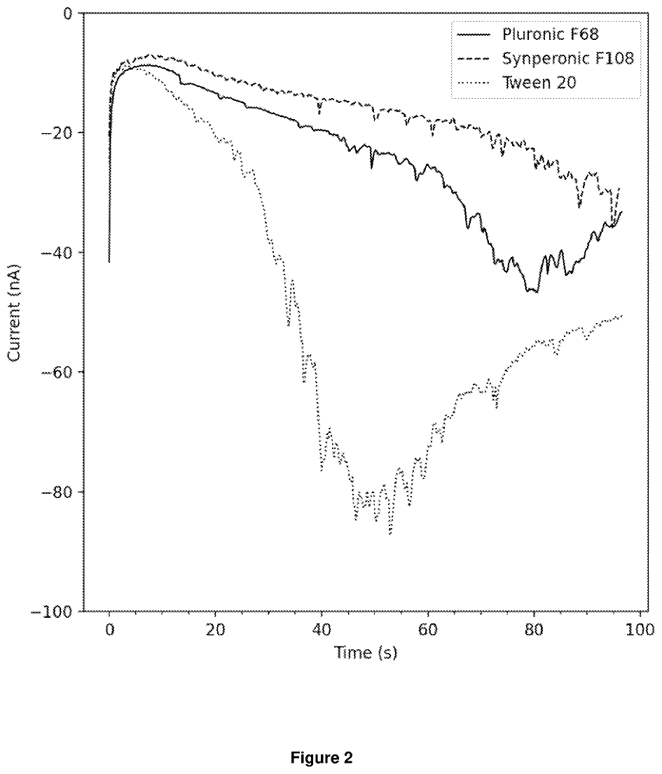

FIG. 2 shows the effect on the quality of the electrochemical signal from adding different surfactants to the wash buffer used to carry out an immunoassay on a microfluidic disposable cartridge. Samples were human whole blood mixed with diluent buffer (25 mL tris buffer, 400 mM NaCl, 16 mM CaCl2, 200 mM guanidine hydrochloride, 1% casein, 0.03% heterophile blocking composition-24), 4 ug/mL detection antibody and magnetic beads coated with capture antibody. This mixture was incubated for several minutes on a cartridge, with fluids manipulated using a device built in-house. The magnetic beads were pulled down onto a magnet and washed with 200 uL tris-buffered saline (TBS) plus 0.05% of different surfactants, given in figure legends. After washing the beads were then transferred to a second sensor using TBS-0.05% surfactant. The magnetic beads were held in place using a second magnet. A chronoamperometry measurement was undertaken using a three-electrode system, with screen-printed carbon used for each of the working, counter and reference electrodes. A voltage of −50 mV was applied for 60s and the current recorded (see FIG. 2).

TABLE 2
Surfactants investigated. Non-ionic surfactants selected from
Surf's Up ® kit from QED Bioscience Inc.

| | Molecular | |
|---|---|---|
| | Weight | |
| Name | (g/mol) | Application |
| Pluronic ® F68 | 8400 | Good solubilizer. |
| Poly(oxyethylene- | | |
| cooxypropylene) block | | |
| copolymer | | |
| Synperonic ® F108 | 14000 | Good emulsifier and solubilizer. |
| Polyethylene glycol- | | |
| polypropylene glycol- | | |
| polyethyleneglycol | | |
| triblock polymer | | |
| Tween ® 20 | 1228 | Non-hemolytic, very hydrophilic, |
| Polyoxyethylene (20) | | good emulsifier and solubilizer. |
| sorbitan monolaurate | | |

### Example 6-Wash Buffers Comprising Block Copolymers

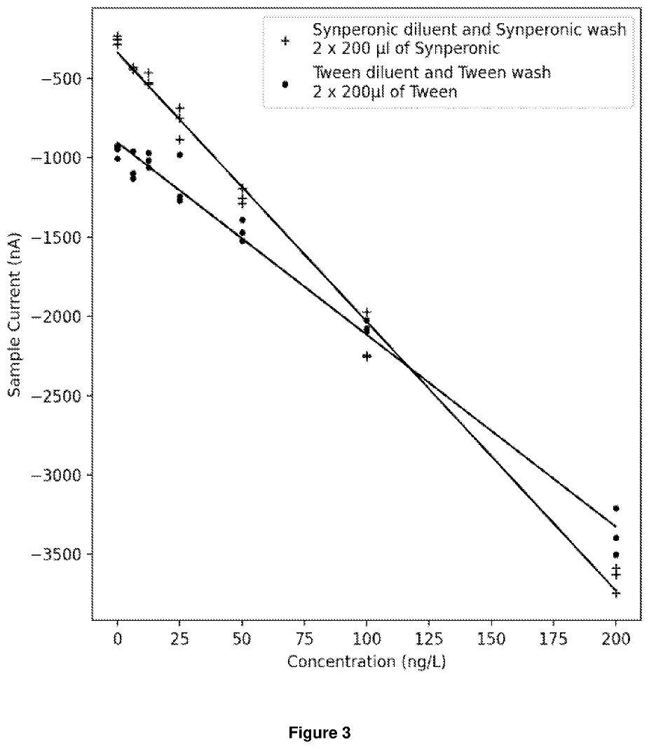

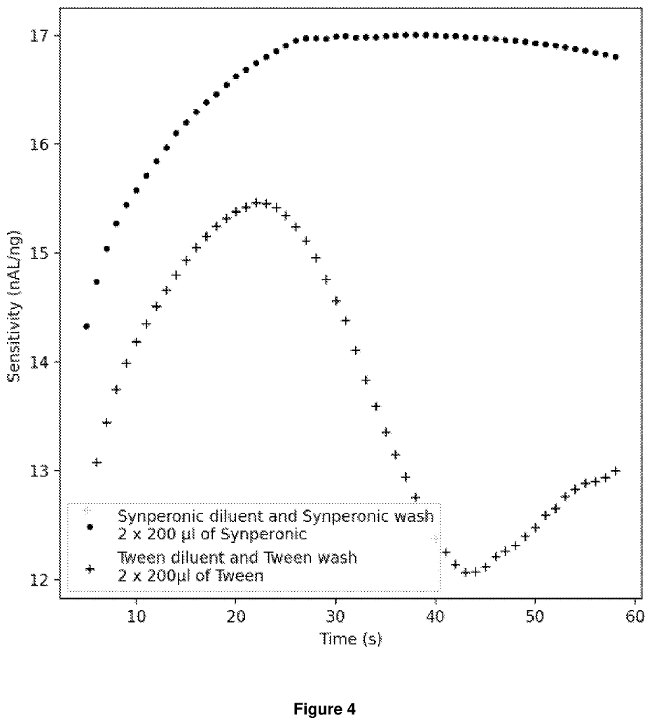

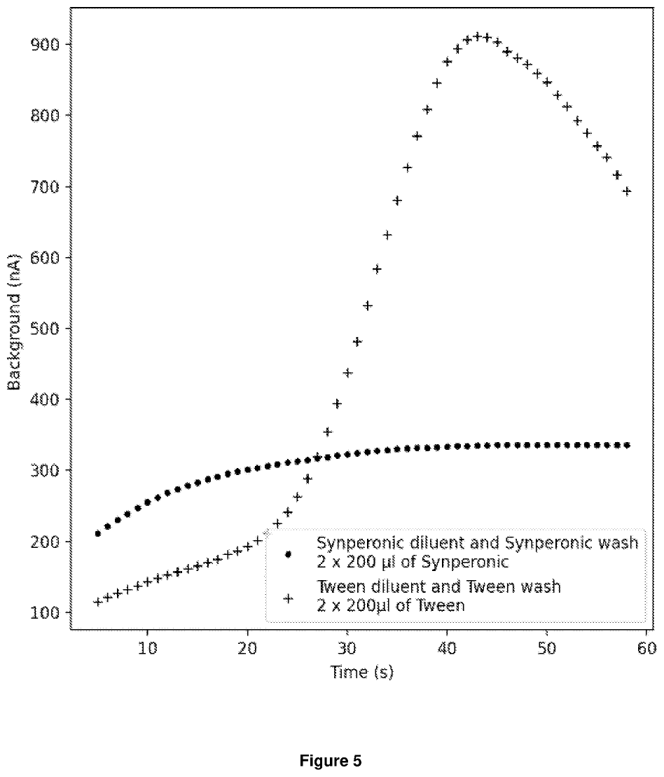

FIGS. 3-5 show the effect on the performance of an immunoassay from adding different surfactants to the wash buffer. Samples were human whole blood mixed with diluent (25 mL tris buffer, 400 mM NaCl, 16 mM CaCl₂), 200 mM guanidine hydrochloride, 1% casein, 0.03% heterophile blocking composition-24), 4 ug/mL detection antibody, 8 ug/mL Horseradish Peroxidase (HRP) and magnetic beads coated with capture antibody. The diluent buffer was spiked with various concentrations of analyte from 0 to 200 pg/mL. These mixtures were incubated for several minutes on a 96 well plate at 37° C. The wells were then emptied of the mixture and washed twice with 100 μL of wash solution, each time retaining the magnetic beads. The wash solution was tris-buffered saline (TBS) mixed with 0.05% of a surfactant, with the surfactant identity shown in the figure legend (see FIGS. 3-5).

After washing the magnetic particles were localised on an 3 mm electrode, and 3,3′,5,5′-tetramethylbenzidine (TMB) solution introduced. A chronoamperometry measurement was undertaken using a three electrode system, with screen-printed carbon used for each of the working, counter and reference electrodes. A voltage of −200 mV was applied for 60s and the current recorded.

TABLE 3
Surfactants investigated. Non-ionic surfactants selected from
Surf's Up ® kit from QED Bioscience Inc.

| | Molecular | |
|---|---|---|
| | Weight | |
| Name | (g/mol) | Application |
| Synperonic ® F108 | 14000 | Good emulsifier and solubilizer. |
| Polyethylene glycol- | | |
| polypropylene glycol- | | |
| polyethyleneglycol | | |
| triblock polymer | | |
| Tween ® 20 | 1228 | Non-hemolytic, very hydrophilic, |
| Polyoxyethylene (20) | | good emulsifier and solubilizer. |
| sorbitan monolaurate | | |

### Example 7-Diluent Buffer Comprising a Blocking Agent and a Block Copolymer

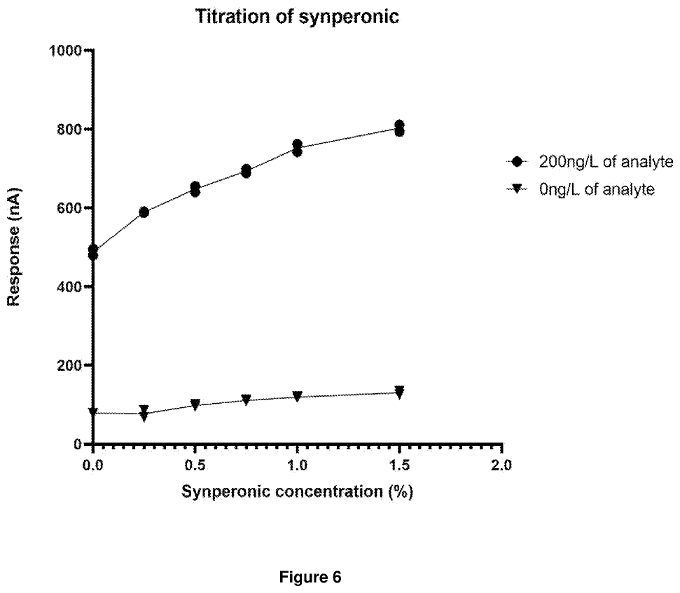

FIG. 6 shows the effect on assay performance from increasing concentrations of Synperonic® in diluent buffer containing bovine serum albumin (BSA). Human plasma (Lithium heparin) containing zero analyte and the same plasma supplemented with 200 ng/L analyte were measured in parallel using a standard procedure in 96-well plate format wherein only the concentration of Synperonic® block copolymer in the sample diluent was varied.

Variations of sample diluent (aqueous) containing identical concentrations of bovine serum albumin (BSA), sodium chloride (NaCl), calcium chloride (CaCl2) any varying concentrations of Synperonic® block copolymer were tested under standardised assay conditions. Plasma samples were mixed with sample diluent containing detection antibody labelled with horseradish peroxidase (HRP) in a 1:1 ratio and magnetic particles coated with capture antibody. The reaction mixture was incubated in a well plate for 8 minutes at 37° C. after which the magnetic particles were removed by magnetic field and washed with assay wash buffer containing a standard concentration of Synperonic® in water. The washed magnetic particles were localised on a 3 mm electrode and 3,3′,5,5′-Tetramethylbenzidine (TMB) solution introduced. The assay response current recorded using chronoamperometry at −200 mV for zero and 200 ng/L analyte samples using each sample diluent (varying in Synperonic®) concentration) showed that increasing Synperonic® concentration in the sample diluent improves the assay sensitivity by increasing the difference in assay response between samples containing zero and 200 ng/L analyte (FIG. 6).

### Example 8-Diluent Buffer Comprising a Blocking Agent and a Block Copolymer

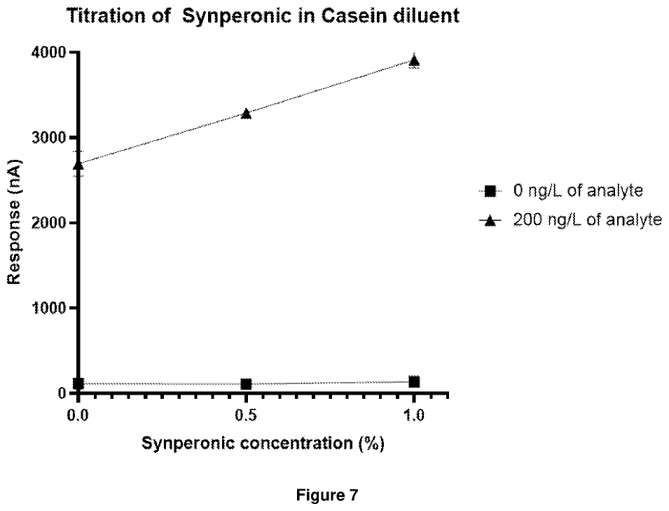

FIG. 7 shows the effect on assay performance from increasing concentrations of Synperonic® in a diluent buffer containing casein. Human plasma (Lithium heparin) containing zero analyte and the same plasma supplemented with 200 ng/L analyte were measured in parallel using a standard procedure in 96-well plate format wherein only the concentration of Synperonic® block copolymer in the sample diluent was varied.

Variations of sample diluent (aqueous) containing identical concentrations of Casein, sodium chloride (NaCl), calcium chloride (CaCl₂)) any varying concentrations of Synperonic® block copolymer were tested under standardised assay conditions. Plasma samples were mixed with sample diluent containing detection antibody labelled with horseradish peroxidase (HRP) in a 1:1 ratio and magnetic particles coated with capture antibody. The reaction mixture was incubated in a well plate for 8 minutes at 37° C. after which the magnetic particles were removed by magnetic field and washed with assay wash buffer containing a standard concentration of Synperonic® in water. The washed magnetic particles were localised on a 3 mm electrode and 3,3′,5,5′-Tetramethylbenzidine (TMB) solution introduced. The assay response current recorded using chronoamperometry at −200 mV for zero and 200 ng/L analyte samples using each sample diluent (varying in Synperonic® concentration) showed that increasing Synperonic® concentration in the sample diluent improves the assay sensitivity by increasing the difference in assay response between samples containing zero and 200 ng/L analyte (FIG. 7).

### Example 9-Effect on the Background from Different Donors of Adding a Chaotropic Agent to the Diluent Buffer

Human blood containing zero analyte was measured using a standard procedure in 96-well plate format wherein only the concentration of Guanidine HCl in the sample diluent was varied.

Variations of sample diluent (25 mM Tris aqueous) containing identical concentrations of casein (1%), 150 mM sodium chloride (NaCl), 16 mM calcium chloride (CaCl₂)) and two concentrations of Guanidine HCl were tested under standardised assay conditions: 0 mM and 100 mM. Plasma samples were mixed with sample diluent containing detection antibody labelled with horseradish peroxidase (HRP) in a 1:1 ratio and magnetic particles coated with capture antibody. The reaction mixture was incubated in a well plate for 8 minutes at 37° C. after which the magnetic particles were removed by magnetic field and washed with a wash buffer containing 0.05% (w/v) of Synperonic®. The washed magnetic particles were localised on a 3 mm electrode and 3,3′,5,5′-Tetramethylbenzidine (TMB) solution introduced. The assay response current recorded at 60s using chronoamperometry at −200 mV using each sample diluent (varying in Guanidine HCl) showed that increasing Guanidine HCl concentration in the sample diluent improves assay sensitivity by decreasing the signal background (Table 2).

TABLE 4
Effect on the background from different donors of adding
guanidine hydrochloride to the diluent buffer

| | | | Decrease in |
|---|---|---|---|
| Sample | 0 mM | 100 mM | background |
| ID | Guanidine HCl | Guanidine HCl | signal (%) |
| A | 89.6 | 64.3 | 72% |
| B | 95.3 | 39.8 | 42% |
| C | 29.0 | 18.4 | 63% |
| D | 47.9 | 26.7 | 56% |
| E | 32.3 | 17.6 | 54% |
| F | 76.9 | 60.2 | 78% |
| G | 28.7 | 19.2 | 67% |
| H | 29.7 | 12.6 | 43% |
| I | 82.4 | 42.2 | 51% |
| J | 38.1 | 24.0 | 63% |
| K | 72.0 | 33.8 | 47% |
| L | 28.9 | 17.5 | 61% |
| M | 66.4 | 36.0 | 54% |
| N | 42.3 | 31.6 | 75% |
| O | 64.7 | 39.5 | 61% |

### Example 10-Calibration of Assay Response to Analyte Using a Diluent Containing a Blocking Agent and a Block Copolymer

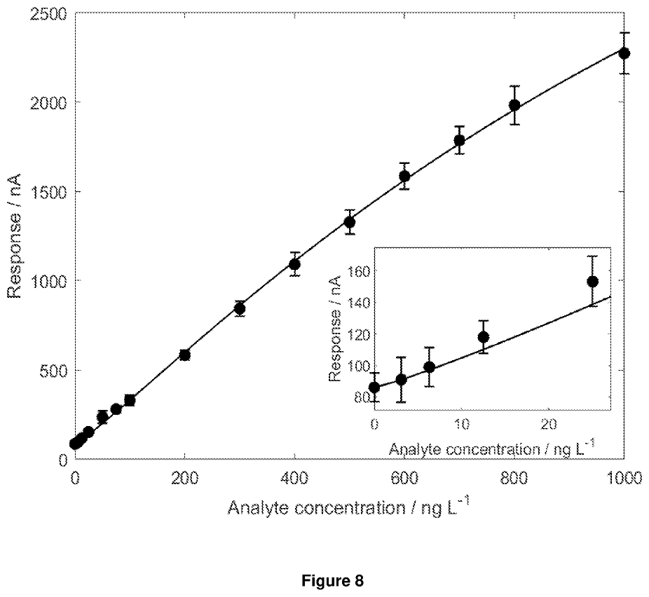

FIG. 8 shows the calibration curve for an immunoassay in a 96 well plate. Human plasma (EDTA) containing zero analyte and the same plasma supplemented with varying concentrations of analyte were measured using a standard procedure.

Magnetic particles coated with capture antibody were mixed with plasma samples and sample diluent (25 mM aqueous TBS) containing bovine serum albumin (5%), sodium chloride (400 mM), calcium chloride (16 mM) and Synperonic® block copolymer (0.25%) and detection antibody labelled with horseradish peroxidase (HRP). The reaction mixture was incubated in a well plate for 5 minutes at 37° C. after which the magnetic particles were removed by magnetic field and washed with assay wash buffer containing a standard concentration of Synperonic® in water. The washed magnetic particles were localised on an electrode and 3,3′,5,5′-Tetramethylbenzidine (TMB) solution introduced. The assay response recorded for each sample (current after 60 seconds chronoamperometry at −200 mV) is plotted against analyte concentration to estimate the sensitivity of the assay response to analyte (FIG. 8).

### Example 11-Calibration of Assay Response to Analyte Using a Diluent Containing a Blocking Agent and a Block Copolymer

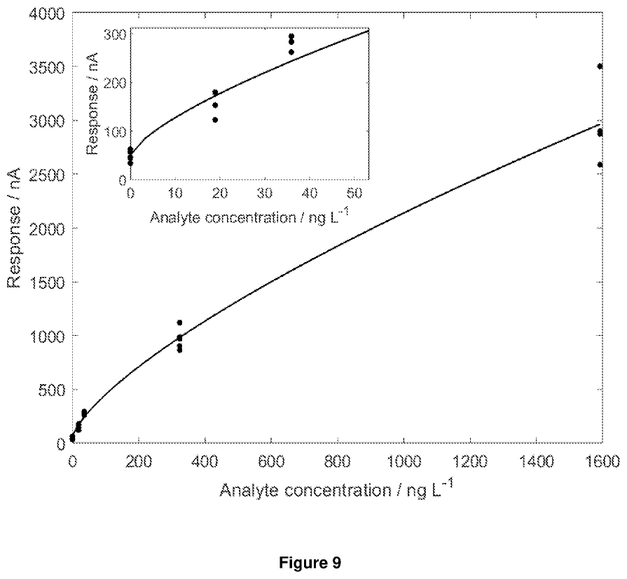

FIG. 9 shows a calibration curve for an immunoassay in a microfluidic disposable cartridge. Human plasma (EDTA) containing zero analyte and the same plasma supplemented with varying concentrations of analyte were measured using a standard procedure.

Magnetic particles coated with capture antibody were mixed with plasma samples and sample diluent (25 mM aqueous TBS) containing bovine serum albumin (5%), sodium chloride (400 mM), calcium chloride (16 mM) and Synperonic® block copolymer (0.25%) and detection antibody labelled with horseradish peroxidase (HRP). The reaction mixture was incubated in a flow channel for 5 minutes at 37° C. after which the magnetic particles were removed by magnetic field and washed with assay wash buffer containing a standard concentration of Synperonic® in water. The washed magnetic particles were localised on an electrode and 3,3′,5,5′-Tetramethylbenzidine (TMB) solution introduced. The assay response recorded for each sample (current after 60 seconds chronoamperometry at −200 mV) is plotted against analyte concentration to estimate the sensitivity of the assay response to analyte (FIG. 9).

### Example 12-Calibration of Assay Response to Analyte Using a Diluent Containing a Blocking Agent and a Block Copolymer

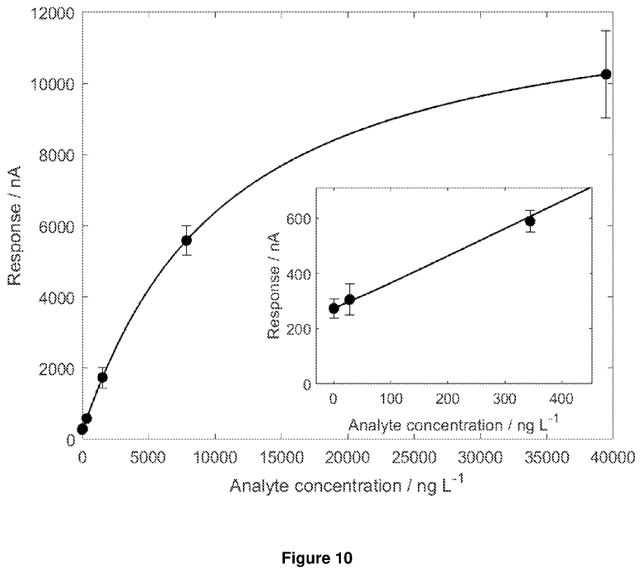

FIG. 10 shows a calibration curve for an immunoassay in a microfluidic disposable cartridge. Human plasma (EDTA) containing zero analyte and the same plasma supplemented with varying concentrations of analyte were measured using a standard procedure.

Magnetic particles coated with capture antibody were mixed with plasma samples and sample diluent (25 mM aqueous TBS) containing casein (0.5%), sodium chloride (150 mM), calcium chloride (16 mM) and Synperonic® block copolymer (1%) and detection antibody labelled with horseradish peroxidase (HRP). The reaction mixture was incubated in a flow channel for 5 minutes at 37° C. after which the magnetic particles were removed by magnetic field and washed with assay wash buffer containing a standard concentration of Synperonic® in water. The washed magnetic particles were localised on an electrode and 3,3′,5,5′-Tetramethylbenzidine (TMB) solution introduced. The assay response recorded for each sample (current after 60 seconds chronoamperometry at −200 mV) is plotted against analyte concentration to estimate the sensitivity of the assay response to analyte.

### Example 13-Production of Calibration Curve for Cardiac Troponin (cTnl) Assay Using Diluent Formed from Lyophilised Components

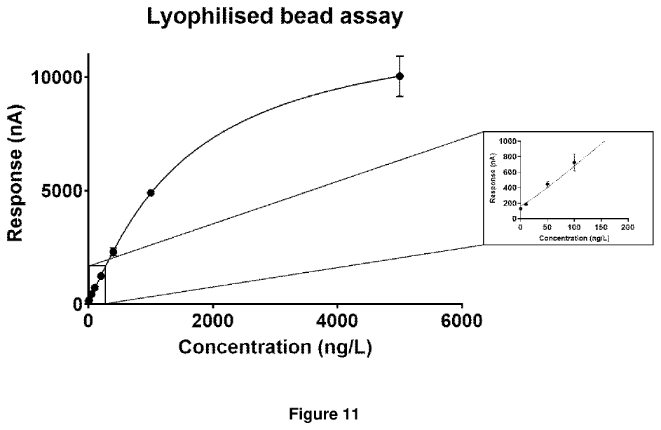

FIG. 11 shows a calibration curve performed on a 96-well plate using samples of known hs-cTnl concentration as follows.

This assay utilised lyophilised beads of reagents. Lyophilised beads 1 contain BSA and magnetic particles coated with capture antibody. Lyophilised beads 2 contain BSA and detection antibodies (anti-cTnl conjugated with HRP). Lyophilised beads 3 contain BSA, heterophile blocking composition and guanidine HCl.

Lyophilised beads 1 and 3 were dissolved with 25 μL assay diluent (of the composition shown in Table 5) to produce magnetic particles coated with capture antibody in an analyte capture diluent. The analyte capture diluent containing magnetic particles was used to dissolve 50 μL of sample to produce a homogenous reaction volume of 75 μL. After 5 minutes the magnetic beads were immobilised using a magnet and washed with 400 μL wash buffer.

50 μL of assay diluent (of the composition shown in Table 5) was used to dissolve lyophilised bead 2 to produce a detection solution containing anti-cTnl detection antibody conjugated with an enzymatic label in an analyte detection diluent. This detection solution was added to the washed magnetic particles for 2.5 minutes before a second wash step of 400 μL wash buffer. Finally, 250 μL TMB solution was added over the magnetic particles to generate an enzyme product which can be detected by an electrode and proportional to analyte concentration. The assay response recorded for each sample (current after 60 seconds chronoamperometry at −200 mV) is plotted against analyte concentration to estimate the sensitivity of the assay response to analyte.

TABLE 5
Assay diluent reagents and concentrations.

| | Component and concentration |
|---|---|
| Assay Diluent | 25 mM Tris |
| | 0.125% Synperonic ® F108 |
| | 275 mM NaCl |
| | 8 mM CaCl₂ |
| | 0.05% Antifoam 204 |
| | 0.05% Proclin ™ 300 |
| Analyte Capture | 2.5% BSA (total provided by lyo beads |
| Diluent (components in | 1 and 3) |
| addition to assay | 200 ug/mL heterophile blocking |
| diluent components) | composition (MAB33 IgG1/IgG1 Poly, |
| | provided by lyo bead 3) |
| | 300 mM Guanidine HCl (provided by lyo |
| | bead 3) |
| Wash buffer | 25 mM Tris |
| | 150 mM NaCl |
| | 0.05% Synperonic ® F108 |
| Analyte Detection Diluent | 2.5% BSA (provided by lyo bead 2) |
| (components in addition | |
| to assay diluent components) | |

The diluents were prepared using Pierce™ 20×Tris Buffered Saline (TBS, obtained from Thermo Fisher), sodium chloride, calcium chloride, Synperonic® F108, Antifoam 204, Proclin™ 300 bovine serum albumin and guanidine hydrochloride (obtained from Sigma-Aldrich) and MAB33 lgG1/lgG1 Poly (obtained from Roche). In the assay diluent, 150 mM NaCl was obtained from the TBS and 125 mM NaCl was added.

A cTnl assay as described above may be performed on a microfluidic cartridge, in which assay reagents may be stored on-cartridge as liquids (for example in liquid blisters) or as dried lyophilised beads.

The embodiments of the invention described herein and shown in the drawings are exemplary embodiments only and are not intended to limit the scope of the appended claims, including any equivalents as included within the scope of the claims. Various modifications are possible and will be readily apparent to the skilled person in the art. It is intended that any combination of non-mutually exclusive features described herein are within the scope of the present invention. That is, features of the described embodiments can be combined with any appropriate aspect described above and optional features of any one aspect can be combined with any other appropriate aspect.

### Further Embodiments of the Present Invention are Described Below

1. A composition for immunoassays comprising a buffer compound, a salt and one or more block copolymers.

2. The composition for immunoassays according to embodiment 1, wherein the buffer compound is selected from the list consisting of: Tris (tris(hydroxymethyl) aminomethane or 2-amino-2-(hydroxymethyl) propane-1,3-diol), TAPS ([tris(hydroxymethyl) methylamino]propanesulfonic acid), bicine (2-(bis(2-hydroxyethyl)amino) acetic acid), tricine (N-[tris(hydroxymethyl) methyl]glycine), TAPSO (3-[N-tris(hydroxymethyl) methylamino]-2-hydroxypropanesulfonic acid), HEPES (4-(2-hydroxyethyl)-1-piperazineethanesulfonic acid), TES (2-[1,3-dihydroxy-2-(hydroxymethyl) propan-2-yl]amino]ethanesulfonic acid), MOPS (3-(N-morpholino) propanesulfonic acid), PIPES (piperazine-N,N′-bis(2-ethanesulfonic acid)), cacodylate (dimethylarsenic acid) and MES (2-(N-morpholino) ethanesulfonic acid).

3. The composition for immunoassays according to embodiment 1 or embodiment 2 wherein the salt is selected from the list consisting of: sodium chloride, aluminium ammonium sulphate, aluminium chloride, aluminium chloride, aluminium fluoride, aluminium nitrate, aluminium potassium sulphate, aluminium sulphate, antimony (III) sulphide, antimony trichloride, barium bromide, barium carbonate, barium chloride, barium chromate, barium nitrate, barium perchlorate, barium sulphate, bismuth carbonate, bismuth nitrate, bismuth oxychloride, bismuth subnitrate, bismuth sulphate, bismuth trichloride, cadmium carbonate, cadmium chloride, cadmium iodide, cadmium nitrate, cadmium oxide, cadmium sulphate, calcium bromide, calcium carbonate, calcium chloride, calcium fluoride, calcium nitrate, calcium oxide powder, calcium sulphate, chromium (III) chloride, chromium (III) nitrate, chromium (III) oxide, chromium piconilate, chromium trioxide, cobalt (II) carbonate, cobalt (II) chloride, cobalt (II) nitrate, cobalt (II) sulphate, cobalt oxide, cupric bromide, cupric carbonate, cupric chloride, cupric nitrate, cupric oxide powder, cupric sulphate, cuprous bromide, cuprous chloride, cuprous iodide, cuprous oxide, dysprosium oxide, europium oxide, ferric chloride, ferric nitrate, ferric oxide, ferric sulphate, ferrous chloride, ferrous sulphate, gadolinium (III) oxide, germanium dioxide, holmium oxide, indium (III) chloride, iridium trichloride, lanthanum carbonate, lanthanum chloride, lanthanum nitrate, lanthanum oxalate, lanthanum oxide, lead (II) chloride, lead acetate, lead bromide, lead carbonate, lead dioxide, lead monoxide, lead nitrate, lead oxide, lead sulphate, magnesium bromide, magnesium nitrate, magnesium oxide, magnesium sulphate, manganese (II) sulphate, manganese carbonate, manganese dioxide, mercuric iodide, mercuric nitrate, mercuric oxide, molybdenum trioxide, neodymium oxide, nickel carbonate, nickel chloride, nickel nitrate, nickel oxide, nickel sulphate, niobium pentoxide, platinum chloride, potassium bromide, potassium carbonate, potassium chloride, potassium chloroplatinate, potassium chromate, potassium iodate, potassium iodide, potassium nitrate, potassium pyroantimonate, rhodium trichloride, rubidium chloride, samarium oxide, selenium dioxide, silver benzoate, silver bromide, silver carbonate, silver chloride, silver iodide, silver nitrate, silver oxide, silver sulphate, sodium arsenate, sodium arsenite, sodium bromide, sodium carbonate, sodium fluoride, sodium iodide, sodium metaborate, sodium oxalate, sodium silicate, sodium silicofluoride, sodium stannate, sodium tellurite, stannic chloride, stannic oxide, stannous chloride, stannous sulphate, strontium carbonate, titanium tetrachloride, tungsten (VI) oxide, vanadium pentoxide, yttrium oxide, zinc chloride, zinc cyanide, zinc nitrate, zinc oxide, zinc sulphate, zirconium dioxide and zirconyl nitrate.

4. The composition for immunoassays according to any preceding embodiment, wherein the buffer compound is at a concentration of about 1 mM to 500 mM.

5. The composition for immunoassays according to any preceding embodiment, comprising Tris (tris(hydroxymethyl) aminomethane or 2-amino-2-(hydroxymethyl) propane-1,3-diol) at a concentration of about 1 mM to about 100 mM.

6. The composition for immunoassays according to any preceding embodiment, comprising Tris (tris(hydroxymethyl) aminomethane or 2-amino-2-(hydroxymethyl) propane-1,3-diol) at a concentration of about 10 mM to about 100 mM.

7. The composition for immunoassays according to any preceding embodiment, comprising Tris (tris(hydroxymethyl) aminomethane or 2-amino-2-(hydroxymethyl) propane-1,3-diol) at a concentration of about 20 mM to about 30 mM.

8. The composition for immunoassays according to any preceding embodiment, comprising Tris 2-amino-2-(tris(hydroxymethyl) aminomethane or (hydroxymethyl) propane-1,3-diol) at a concentration of about 25 mM.

9. The composition for immunoassays according to any preceding embodiment, wherein the salt is at a concentration of about 1 mM to about 1000 mM.

10. The composition for immunoassays according to any preceding embodiment, comprising sodium chloride (NaCl) at a concentration of about 1 mM to about 1000 mM.

11. The composition for immunoassays according to any preceding embodiment, comprising sodium chloride (NaCl) at a concentration of about 50 to about 500 mM.

12. The composition for immunoassays according to any preceding embodiment, comprising sodium chloride (NaCl) at a concentration of about 200 to about 400 mM.

13. The composition for immunoassays according to any preceding embodiment, comprising sodium chloride (NaCl) at a concentration of about 100 to about 200 mM.

14. The composition for immunoassays according to any one of embodiments 1 to 11 and 13, comprising sodium chloride (NaCl) at a concentration of about 150 mM.

15. The composition for immunoassays according to any one of embodiments 1 to 12, comprising sodium chloride (NaCl) at a concentration of about 400 mM.

16. The composition for immunoassays according to any one of embodiments 1 to 12, comprising sodium chloride (NaCl) at a concentration of about 275 mM.

17. The composition for immunoassays according to any preceding embodiment comprising Tris (tris(hydroxymethyl) aminomethane or 2-amino-2-(hydroxymethyl) propane-1,3-diol), sodium chloride and one or more block copolymers.

18. The composition for immunoassays according to any preceding embodiment comprising tris-buffered saline (TBS).

19. The composition for immunoassays according to embodiment 18, wherein the tris-buffered saline (TBS) comprises Tris (tris(hydroxymethyl) aminomethane or 2-amino-2-(hydroxymethyl) propane-1,3-diol) at a concentration of about 20 mM to about 30 mM and comprises NaCl at a concentration of about 100 mM to about 200 mM.

20. The composition for immunoassays according to embodiment 19, wherein the tris-buffered saline (TBS) comprises Tris (tris(hydroxymethyl) aminomethane or 2-amino-2-(hydroxymethyl) propane-1,3-diol) at a concentration of about 25 mM and comprises NaCl at a concentration of about 150 mM.

21. The composition for immunoassays according to embodiment 18, wherein the tris-buffered saline (TBS) comprises Tris (tris(hydroxymethyl) aminomethane or 2-amino-2-(hydroxymethyl) propane-1,3-diol) at a concentration of about 20 mM to about 30 mM and comprises NaCl at a concentration of about 50 mM to about 500 mM.

22. The composition for immunoassays according to embodiment 21, wherein the tris-buffered saline (TBS) comprises Tris (tris(hydroxymethyl) aminomethane or 2-amino-2-(hydroxymethyl) propane-1,3-diol) at a concentration of about 25 mM and comprises NaCl at a concentration of about 400 mM.

23. The composition for immunoassays according to any one of embodiments 1 to 13 and 15 to 22, wherein the composition comprises tris-buffered saline (TBS) and additional NaCl, to provide a composition comprising Tris (tris(hydroxymethyl) aminomethane or 2-amino-2-(hydroxymethyl) propane-1,3-diol) at a concentration of about 20 mM to about 30 mM and NaCl at a total concentration of about 200 mM to about 500 mM.

24. The composition for immunoassays according to embodiment 23, wherein the composition comprises tris-buffered saline (TBS) and additional NaCl, to provide a composition with comprising Tris (tris(hydroxymethyl) aminomethane or 2-amino-2-(hydroxymethyl) propane-1,3-diol) at a concentration of about 20 mM to about 30 mM and NaCl at a total concentration of about 200 mM to about 400 mM.

25. The composition for immunoassays according to embodiment 24, wherein the composition comprises Tris (tris(hydroxymethyl) aminomethane or 2-amino-2-(hydroxymethyl) propane-1,3-diol) at a concentration of about 25 mM and NaCl at a total concentration of about 275 mM.

26. The composition for immunoassays according to any preceding embodiment, comprising one or more block copolymers at a total concentration of about 0.01% (w/v) to about 2% (w/v).

27. The composition for immunoassays according to any preceding embodiment, comprising one or more block copolymers at a total concentration of about 0.25% (w/v) to about 1.5% (w/v).

28. The composition for immunoassays according to any preceding embodiment, comprising one or more block copolymers at a total concentration of about 0.05% (w/v).

29. The composition for immunoassays according to any preceding embodiment, comprising one or more block copolymers at a total concentration of about 0.125% (w/v).

30. The composition for immunoassays according to any preceding embodiment, comprising Tris (tris(hydroxymethyl) aminomethane or 2-amino-2-(hydroxymethyl) propane-1,3-diol) at a concentration of about 1 mM to about 500 mM, sodium chloride (NaCl) at a concentration of about 1 mM to about 1000 mM and one or more block copolymers at a total concentration of about 0.01% (w/v) to about 2% (w/v).

31. The composition for immunoassays according to any preceding embodiment comprising Tris (tris(hydroxymethyl) aminomethane or 2-amino-2-(hydroxymethyl) propane-1,3-diol) at a concentration of 25 mM, sodium chloride (NaCl) at a concentration of 150 mM and a block copolymer at a concentration of 0.05% (w/v).

32. The composition for immunoassays according to any one of embodiments 1 to 30, comprising Tris (tris(hydroxymethyl) aminomethane or 2-amino-2-(hydroxymethyl) propane-1,3-diol) at a concentration of 25 mM, sodium chloride (NaCl) at a concentration of 275 mM and a block copolymer at a concentration of 0.125% (w/v).

33. A composition for immunoassays comprising a buffer compound, one or more salts, one or more blocking agents and one or more block copolymers.

34. A composition for immunoassays according to embodiment 33, wherein the one or more blocking agents are selected from the list consisting of: proteins, such as phosphoproteins, polymers and small molecules.

35. A composition for immunoassays according to embodiment 34, wherein the protein is a phosphoprotein, optionally wherein the phosphoprotein is casein.

36. A composition for immunoassays according to embodiment 34, wherein the protein is bovine serum albumin (BSA).

37. A composition for immunoassays according to embodiment 34, wherein the polymer is PVP (polyvinylpyrrolidone) or PVA (polyvinyl alcohol).

38. A composition for immunoassays according to embodiment 34, wherein the small molecule is ethanolamine.

39. A composition for immunoassays according to any one of embodiments 33 to 38 comprising at least two salts, for example two, three, four, five or six salts.

40. A composition for immunoassays according to any one of embodiments 33 to 39 comprising two salts.

41. The composition for immunoassays according to any one of embodiments 33 to 40 wherein the buffer compound is selected from the list consisting of: Tris (tris(hydroxymethyl) aminomethane or 2-amino-2-(hydroxymethyl) propane-1,3-diol), TAPS ([tris(hydroxymethyl) methylamino]propanesulfonic acid), bicine (2-(bis(2-hydroxyethyl)amino) acetic acid), tricine (N-[tris(hydroxymethyl) methyl]glycine), TAPSO (3-[N-tris(hydroxymethyl) methylamino]-2-hydroxypropanesulfonic acid), HEPES (4-(2-hydroxyethyl)-1-piperazineethanesulfonic acid), TES (2-[1,3-dihydroxy-2-(hydroxymethyl) propan-2-yl]amino]ethanesulfonic acid), MOPS (3-(N-morpholino) propanesulfonic acid), PIPES (piperazine-N,N′-bis(2-ethanesulfonic acid)), cacodylate (dimethylarsenic acid) and MES (2-(N-morpholino) ethanesulfonic acid)

42. The composition for immunoassays according to any one of embodiments 33 to 41 wherein the one or more salts are selected from the list consisting of: sodium chloride, calcium chloride, aluminium ammonium sulphate, aluminium chloride, aluminium chloride, aluminium fluoride, aluminium nitrate, aluminium potassium sulphate, aluminium sulphate, antimony (III) sulphide, antimony trichloride, barium bromide, barium carbonate, barium chloride, barium chromate, barium nitrate, barium perchlorate, barium sulphate, bismuth carbonate, bismuth nitrate, bismuth oxychloride, bismuth subnitrate, bismuth sulphate, bismuth trichloride, cadmium carbonate, cadmium chloride, cadmium iodide, cadmium nitrate, cadmium oxide, cadmium sulphate, calcium bromide, calcium carbonate, calcium fluoride, calcium nitrate, calcium oxide powder, calcium sulphate, chromium (III) chloride, chromium (III) nitrate, chromium (III) oxide, chromium piconilate, chromium trioxide, cobalt (II) carbonate, cobalt (II) chloride, cobalt (II) nitrate, cobalt (II) sulphate, cobalt oxide, cupric bromide, cupric carbonate, cupric chloride, cupric nitrate, cupric oxide powder, cupric sulphate, cuprous bromide, cuprous chloride, cuprous iodide, cuprous oxide, dysprosium oxide, europium oxide, ferric chloride, ferric nitrate, ferric oxide, ferric sulphate, ferrous chloride, ferrous sulphate, gadolinium (III) oxide, germanium dioxide, holmium oxide, indium (III) chloride, iridium trichloride, lanthanum carbonate, lanthanum chloride, lanthanum nitrate, lanthanum oxalate, lanthanum oxide, lead (II) chloride, lead acetate, lead bromide, lead carbonate, lead dioxide, lead monoxide, lead nitrate, lead oxide, lead sulphate, magnesium bromide, magnesium nitrate, magnesium oxide, magnesium sulphate, manganese (II) sulphate, manganese carbonate, manganese dioxide, mercuric iodide, mercuric nitrate, mercuric oxide, molybdenum trioxide, neodymium oxide, nickel carbonate, nickel chloride, nickel nitrate, nickel oxide, nickel sulphate, niobium pentoxide, platinum chloride, potassium bromide, potassium carbonate, potassium chloride, potassium chloroplatinate, potassium chromate, potassium iodate, potassium iodide, potassium nitrate, potassium pyroantimonate, rhodium trichloride, rubidium chloride, samarium oxide, selenium dioxide, silver benzoate, silver bromide, silver carbonate, silver chloride, silver iodide, silver nitrate, silver oxide, silver sulphate, sodium arsenate, sodium arsenite, sodium bromide, sodium carbonate, sodium fluoride, sodium iodide, sodium metaborate, sodium oxalate, sodium silicate, sodium silicofluoride, sodium stannate, sodium tellurite, stannic chloride, stannic oxide, stannous chloride, stannous sulphate, strontium carbonate, titanium tetrachloride, tungsten (VI) oxide, vanadium pentoxide, yttrium oxide, zinc chloride, zinc cyanide, zinc nitrate, zinc oxide, zinc sulphate, zirconium dioxide and zirconyl nitrate.

43. The composition for immunoassays according to any one of embodiments 33 to 42, wherein the buffer compound is at a concentration of about 1 mM to 500 mM.

44. The composition for immunoassays according to any one of embodiments 33 to 43, comprising Tris (tris(hydroxymethyl) aminomethane or 2-amino-2-(hydroxymethyl) propane-1,3-diol) at a concentration of about 1 mM to about 100 mM.

45. The composition for immunoassays according to any one of embodiments 33 to 44, comprising Tris (tris(hydroxymethyl) aminomethane or 2-amino-2-(hydroxymethyl) propane-1,3-diol) at a concentration of about 20 mM to about 30 mM.

46. The composition for immunoassays according to any one of embodiments 33 to 45, comprising Tris (tris(hydroxymethyl) aminomethane or 2-amino-2-(hydroxymethyl) propane-1,3-diol) at a concentration of about 25 mM.

47. The composition for immunoassays according to any one of embodiments 33 to 46, wherein one of the one or more salts is at a concentration of about 1 mM to about 1000 mM.

48. The composition for immunoassays according to any one of embodiments 33 to 47, comprising sodium chloride (NaCl) at a concentration of about 1 mM to about 1000 mM.

49. The composition for immunoassays according to any one of embodiments 33 to 48, comprising sodium chloride (NaCl) at a concentration of about 200 mM to about 400 mM.

50. The composition for immunoassays according to any one of embodiments 33 to 48, comprising sodium chloride (NaCl) at a concentration of about 300 mM to about 500 mM.

51. The composition for immunoassays according to any one of embodiments 33 to 48, comprising sodium chloride (NaCl) at a concentration of about 100 mM to about 200 mM.

52. The composition for immunoassays according to embodiment 48, comprising sodium chloride (NaCl) at a concentration of about 400 mM.

53. The composition for immunoassays according to embodiment 48, comprising sodium chloride (NaCl) at a concentration of about 275 mM.

54. The composition for immunoassays according to embodiment 48, comprising sodium chloride (NaCl) at a concentration of about 150 mM.

55. The composition for immunoassays according to any one of embodiments 33 to 54, wherein one of the one or more salts is at a concentration of about 1 mM to about 200 mM.

56. The composition for immunoassays according to any one of embodiments 33 to 55, comprising calcium chloride (CaCl₂)) at a concentration of about 1 mM to about 200 mM.

57. The composition for immunoassays according to any one of embodiments 33 to 56, comprising calcium chloride (CaCl₂)) at a concentration of about 5 mM to about 20 mM.

58. The composition for immunoassays according to any one of embodiments 33 to 57, comprising calcium chloride (CaCl₂)) at a concentration of about 10 mM to about 20 mM.

59. The composition for immunoassays according to embodiment 58, comprising calcium chloride (CaCl₂)) at a concentration of about 16 mM.

60. The composition for immunoassays according to embodiment 57, comprising calcium chloride (CaCl₂)) at a concentration of about 8 mM.

61. The composition for immunoassays according to any one of embodiments 33 to 60 comprising tris-buffered saline (TBS).

62. The composition for immunoassays according to any one of embodiments 33 to 61, wherein the composition comprises Tris (tris(hydroxymethyl) aminomethane or 2-amino-2-(hydroxymethyl) propane-1,3-diol) at a concentration of about 20 mM to about 30 mM and comprises NaCl at a concentration of about 100 mM to 200 mM.

63. The composition for immunoassays according to embodiment 62, wherein the composition comprises Tris (tris(hydroxymethyl) aminomethane or 2-amino-2-(hydroxymethyl) propane-1,3-diol) at a concentration of about 25 mM and comprises NaCl at a concentration of about 150 mM.

64. The composition for immunoassays according to any one of embodiments 33 to 61, wherein the composition comprises Tris (tris(hydroxymethyl) aminomethane or 2-amino-2-(hydroxymethyl) propane-1,3-diol) at a concentration of about 20 mM to about 30 mM and comprises NaCl at a concentration of about 200 mM to 400 mM.

65. The composition for immunoassays according to embodiment 64, wherein the composition comprises Tris (tris(hydroxymethyl) aminomethane or 2-amino-2-(hydroxymethyl) propane-1,3-diol) at a concentration of about 25 mM and comprises NaCl at a concentration of about 275 mM.

66. The composition for immunoassays according to any one of embodiments 33 to 61, wherein the composition comprises Tris (tris(hydroxymethyl) aminomethane or 2-amino-2-(hydroxymethyl) propane-1,3-diol) at a concentration of about 20 mM to about 30 mM, NaCl at a concentration of about 200 mM to about 400 mM and CaCl₂) at a concentration of about 5 mM to about 20 mM.

67. The composition for immunoassays according to embodiment 66, wherein the composition comprises Tris (tris(hydroxymethyl) aminomethane or 2-amino-2-(hydroxymethyl) propane-1,3-diol) at a concentration of about 25 mM, NaCl at a concentration of about 275 mM and CaCl₂) at a concentration of about 8 mM.

68. The composition for immunoassays according to any one of embodiments 33 to 61, wherein the composition comprises Tris (tris(hydroxymethyl) aminomethane or 2-amino-2-(hydroxymethyl) propane-1,3-diol) at a concentration of about 20 mM to about 30 mM, NaCl at a concentration of about 300 mM to about 500 mM and CaCl₂) at a concentration of about 10 mM to about 20 mM.

69. The composition for immunoassays according to embodiment 68, wherein the composition comprises Tris (tris(hydroxymethyl) aminomethane or 2-amino-2-(hydroxymethyl) propane-1,3-diol) at a concentration of about 25 mM, NaCl at a concentration of about 400 mM and CaCl₂) at a concentration of about 16 mM.

70. The composition for immunoassays according to any one of embodiments 33 to 69, comprising one or more block copolymers at a total concentration of about 0.05% (w/v) to about 5% (w/v).

71. The composition for immunoassays according to any one of embodiments 33 to 70, comprising one or more block copolymers at a total concentration of about 0.75% (w/v) to about 1.5% (w/v).

72. The composition for immunoassays according to any one of embodiments 33 to 71, comprising one or more block copolymers at a total concentration of about 1% (w/v).

73. The composition for immunoassays according to any one of embodiments 33 to 72, comprising one block copolymer at a concentration of about 1% (w/v).

74. The composition for immunoassays according to any one of embodiments 33 to 70, comprising one or more block copolymers at a total concentration of about 0.05% (w/v) to about 0.5% (w/v).

75. The composition for immunoassays according to embodiment 74, comprising one or more block copolymers at a total concentration of about 0.25% (w/v).

76. The composition for immunoassays according to embodiment 75, comprising one block copolymer at a concentration of about 0.25% (w/v).

77. The composition for immunoassays according to embodiment 70, comprising one or more block copolymers at a total concentration of about 0.125% (w/v).

78. The composition for immunoassays according to embodiment 77, comprising one block copolymer at a concentration of about 0.125% (w/v).

79. The composition for immunoassays according to any one of embodiments 33 to 78, comprising one or more blocking agents at a total concentration of about 0.05% (w/v) to about 10% (w/v), for example about 0.05% (w/v) to about 9% (w/v), about 0.05% (w/v) to about 8% (w/v), about 0.05% (w/v) to about 7% (w/v), about 0.05% (w/v) to about 6% (w/v), about 0.05% (w/v) to about 5% (w/v), about 0.05% (w/v) to about 4% (w/v), about 0.05% (w/v) to about 3% (w/v), about 0.05% (w/v) to about 2.5% (w/v), about 0.05% (w/v) to about 2% (w/v), about 0.05% (w/v) to about 1.5% (w/v), about 0.05% (w/v) to about 1% (w/v), about 0.05% (w/v) to about 0.5% (w/v), about 0.05% (w/v) to about 0.4% (w/v), about 0.05% (w/v) to about 0.3% (w/v), about 0.05% (w/v) to about 0.2% (w/v) or about 0.05% (w/v) to about 0.1% (w/v).

80. The composition for immunoassays according to embodiment 79, comprising a blocking agent comprising bovine serum albumin at a concentration of about 0.05% (w/v) to about 10% (w/v).

81. The composition for immunoassays according to embodiment 80, comprising a blocking agent comprising bovine serum albumin at a concentration of about 0.5% (w/v).

82. The composition for immunoassays according to embodiment 80, comprising a blocking agent comprising bovine serum albumin at a concentration of about 2.5% (w/v).

83. The composition for immunoassays any one of embodiments 33 to 82, comprising Tris (tris(hydroxymethyl) aminomethane or 2-amino-2-(hydroxymethyl) propane-1,3-diol) at a concentration of about 1 mM to about 500 mM, sodium chloride (NaCl) at a concentration of about 1 mM to about 1000 mM, calcium chloride (CaCl₂)) at a concentration of about 1 mM to 200 mM, bovine serum albumin (BSA) at a concentration of about 0.05% (w/v) to about 10% (w/v) and one or more block copolymers at a total concentration of about 0.05% (w/v) to about 5% (w/V).

84. The composition for immunoassays according to embodiment 80, comprising Tris (tris(hydroxymethyl) aminomethane or 2-amino-2-(hydroxymethyl) propane-1,3-diol) at a concentration of 25 mM, sodium chloride (NaCl) at a concentration of 400 mM, calcium chloride (CaCl₂)) at a concentration of 16 mM, bovine serum albumin (BSA) at a concentration of 5% (w/v) and a polyoxyethylene-polyoxypropylene block copolymer (such as Synperonic® F 108) at a concentration of about 0.25% (w/v).

85. The composition for immunoassays according to embodiment 80, comprising Tris (tris(hydroxymethyl) aminomethane or 2-amino-2-(hydroxymethyl) propane-1,3-diol) at a concentration of 25 mM, sodium chloride (NaCl) at a concentration of 275 mM, calcium chloride (CaCl₂)) at a concentration of 8 mM, bovine serum albumin (BSA) at a concentration of 2.5% (w/v) and a polyoxyethylene-polyoxypropylene block copolymer (such as Synperonic® F 108) at a concentration of about 0.125% (w/v).

86. The composition for immunoassays according to any one of embodiments 33 to 79, comprising casein at a concentration of about 0.1% (w/v) to about 3% (w/v).

87. The composition for immunoassays according to embodiment 86, comprising casein at a concentration of about 0.4% (w/v) to about 0.6% (w/v).

88. The composition for immunoassays according to embodiment 86 or embodiment 87, comprising casein at a concentration of about 0.5% (w/v).

89. A composition for immunoassays according to any one of embodiments 33 to 88 comprising Tris (tris(hydroxymethyl) aminomethane or 2-amino-2-(hydroxymethyl) propane-1,3-diol), sodium chloride (NaCl), calcium chloride (CaCl₂)), casein and one or more block copolymers.

90. The composition for immunoassays according to embodiment 89, comprising Tris (tris(hydroxymethyl) aminomethane or 2-amino-2-(hydroxymethyl) propane-1,3-diol) at a concentration of about 1 mM to 500 mM, sodium chloride (NaCl) at a concentration of about 1 mM to 1000 mM, calcium chloride (CaCl₂)) at a concentration of about 1 mM to 200 mM, casein at a concentration of about 0.1% (w/v) to about 2% (w/v) and one or more block copolymers at a total concentration of about 0.05% (w/v) to about 5% (w/v).

91. The composition for immunoassays according to embodiment 90, comprising Tris (tris(hydroxymethyl) aminomethane or 2-amino-2-(hydroxymethyl) propane-1,3-diol) at a concentration of about 25 mM, sodium chloride (NaCl) at a concentration of about 150 mM, calcium chloride (CaCl₂)) at a concentration of about 16 mM, casein at a concentration of about 0.5% (w/v) and a polyoxyethylene-polyoxypropylene block copolymer (such as Synperonic® F 108) at a concentration of about 1% (w/v).

92. The composition for immunoassays according to any preceding embodiment, wherein the block copolymer is a polyoxyethylene-polyoxypropylene block copolymer.

93. The composition for immunoassays according to embodiment 92, wherein the polyoxyethylene-polyoxypropylene block copolymer is represented by Formula I

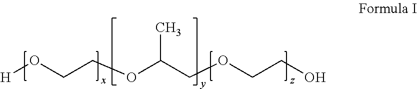

wherein x and z=2-130 and b=15-67.

94. The composition for immunoassays according to embodiment 93, wherein x and z=120-130 and b=50-60.

95. The composition for immunoassays according to any preceding embodiment, wherein the block copolymer is Synperonic® F 108 having the following formula:

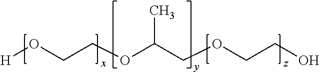

96. The composition for immunoassays according to any preceding embodiment further comprising one or more chaotropic agents.

97. The composition for immunoassays according to any preceding embodiment further comprising one or more denaturing agents.

98. The composition for immunoassays according to any preceding embodiment further comprising one or more chaotropic and denaturing agents.

99. The composition for immunoassays according to any one of embodiments 96 to 98 wherein the one or more chaotropic and/or denaturing agents are selected from a list consisting of: a guanidine salt, urea or betaine.

100. The composition for immunoassays according to embodiment 99, wherein the one or more chaotropic and/or denaturing agents is guanidine hydrochloride.

101. The composition for immunoassays according to any one of embodiments 96 to 100 wherein the one or more chaotropic and/or denaturing agents are at a concentration of about 1 mM to about 1000 mM.

102. The composition for immunoassays any one of embodiments 96 to 101, comprising Tris (tris(hydroxymethyl) aminomethane or 2-amino-2-(hydroxymethyl) propane-1,3-diol) at a concentration of about 1 mM to about 500 mM, sodium chloride (NaCl) at a concentration of about 1 mM to about 1000 mM, calcium chloride (CaCl₂)) at a concentration of about 1 mM to about 200 mM, casein at a concentration of about 0.1% (w/v) to about 2% (w/v), one or more block copolymers at a total concentration of about 0.05% (w/v) to about 5% (w/v) and one or more chaotropic and/or denaturing agents at a concentration of about 1 mM to about 1000 mM.

103. The composition for immunoassays according to embodiment 102, comprising Tris (tris(hydroxymethyl) aminomethane or 2-amino-2-(hydroxymethyl) propane-1,3-diol) at a concentration of about 25 mM, sodium chloride (NaCl) at a concentration of about 150 mM, calcium chloride (CaCl₂)) at a concentration of about 16 mM, casein at a concentration of about 0.5% (w/v), a polyoxyethylene-polyoxypropylene block copolymer (such as Synperonic® F 108) at a concentration of about 1% (w/v) and guanidine hydrochloride (guanidine HCl) at a concentration of about 200 mM.

104. The composition for immunoassays any one of embodiments 96 to 101, comprising Tris (tris(hydroxymethyl) aminomethane or 2-amino-2-(hydroxymethyl) propane-1,3-diol) at a concentration of about 1 mM to about 500 mM, sodium chloride (NaCl) at a concentration of about 1 mM to about 1000 mM, calcium chloride (CaCl₂)) at a concentration of about 1 mM to about 200 mM, bovine serum albumin at a concentration of about 0.5% (w/v) to about 10% (w/v), one or more block copolymers at a total concentration of about 0.05% (w/v) to about 5% (w/v) and one or more chaotropic and/or denaturing agents at a concentration of about 1 mM to about 1000 mM.

105. The composition for immunoassays according to embodiment 104, comprising Tris (tris(hydroxymethyl) aminomethane or 2-amino-2-(hydroxymethyl) propane-1,3-diol) at a concentration of about 25 mM, sodium chloride (NaCl) at a concentration of about 275 mM, calcium chloride (CaCl₂)) at a concentration of about 8 mM, bovine serum albumin at a concentration of about 2.5% (w/v), a polyoxyethylene-polyoxypropylene block copolymer (such as Synperonic® F 108) at a concentration of about 0.125% (w/V) and guanidine hydrochloride (guanidine HCl) at a concentration of about 300 mM.

106. A composition for immunoassays comprising one or more chaotropic and/or denaturing agents.

107. The composition for immunoassays according to embodiment 106, wherein the one or more chaotropic and/or denaturing agents are selected from a list consisting of: a guanidine salt, urea or betaine.

108. The composition for immunoassays according to embodiment 107, wherein the one or more chaotropic and/or denaturing agents is guanidine hydrochloride.

109. The composition for immunoassays according to any one of embodiments 106 to 108, wherein the one or more chaotropic and/or denaturing agents are at a concentration of about 1 mM to about 1000 mM.

110. The composition for immunoassays according to any one of embodiments 106 to 109, further comprising a proteinaceous matrix, optionally wherein the proteinaceous matrix comprises bovine serum albumin (BSA) and/or casein.

111. The composition for immunoassays according to any one of the preceding embodiments wherein the composition further comprises a biocide.

112. The composition for immunoassays according to embodiment 111, wherein the biocide comprises 2-methyl-4-isothiazolin-3-one (MIT), 5-chloro-2-methyl-4-isothiazolin-3-one (CMIT), or a mixture thereof (for example a ProClin™ biocide, such as ProClin™ 300).

113. The composition for immunoassays according to embodiment 111 or 112, wherein the composition comprises the biocide at a concentration of about 0.01% (w/v) to about 2% (w/v), for example about 0.01% (w/v) to about 1% (w/v), about 0.01% (w/v) to about 0.5% (w/v), about 0.01% (w/v) to about 0.1% (w/v), or about 0.02% (w/v) to about 0.08% (w/v).

114. The composition for immunoassays according to embodiment 113, wherein the composition comprises the biocide at a concentration of about 0.03% (w/v) to about 0.07% (w/v), or about 0.05% (w/v).

115. The composition for immunoassays according to any one of the preceding embodiments wherein the composition further comprises an anti-foaming agent 116. The composition for immunoassays according to embodiment 115, wherein the anti-foaming agent comprises non-silicone defoamers in a polyol-based dispersion (for example Antifoam 204).

117. The composition for immunoassays according to embodiment 115 or 116, wherein the composition comprises the anti-foaming agent at a concentration of about 0.01% (w/v) to about 2% (w/v), for example about 0.01% (w/v) to about 1% (w/v), about 0.01% (w/v) to about 0.5% (w/v), about 0.01% (w/v) to about 0.1% (w/v), or about 0.02% (w/v) to about 0.08% (w/v).

118. The composition for immunoassays according to embodiment 117, wherein the composition comprises the anti-foaming agent at a concentration of about 0.03% (w/v) to about 0.07% (w/v), or about 0.05% (w/v).

119. The composition for immunoassays according to any one of the preceding embodiments, wherein the composition further comprises a heterophile blocking composition.

120. The composition for immunoassays according to any one of the preceding embodiments, wherein the composition is a liquid, a gel or an emulsion.

121. The composition for immunoassays according to any one of the preceding embodiments, wherein the composition comprises water.

122. The composition for immunoassays according to any one of the preceding embodiments, wherein the composition is a liquid composition comprising the components of the composition in an aqueous solution, suspension, or combination thereof.

123. The composition for immunoassays according to any one of embodiments 1 to 119, wherein the composition is lyophilised.

124. A method for preparing a composition for immunoassays according to any one of embodiments 1 to 122, wherein the method comprises mixing water with additional components of the composition to provide the composition.

125. The method according to embodiment 124, wherein the method comprises mixing aqueous buffer solution or water with additional components of the composition to provide the composition.

126. The method according to embodiment 124 or 125, wherein the additional components are provided in solid form, in aqueous solution, or a combination thereof.

127. The method according to embodiment 126, wherein one or more of the additional components are provided in lyophilised form prior to mixing with the aqueous buffer solution or water.

128. A method for preparing a composition for immunoassays according to any one of embodiments 124 to 127, wherein the composition comprises a blocking agent, the method comprising:
providing a liquid composition comprising a buffer compound, a salt, one or more block copolymers and optionally one or more additional components of the composition;
providing lyophilised blocking agent; and
mixing the liquid composition with the lyophilised blocking agent to provide the composition for immunoassays.

129. The method according to embodiment 128, wherein the composition further comprises one or more chaotropic and/or denaturing agents and wherein the method further comprises providing the one or more chaotropic and/or denaturing agents in lyophilised form and mixing the liquid composition with the lyophilised blocking agent and the lyophilised one or more chaotropic and/or denaturing agents to provide the composition for immunoassays.

130. The method according to embodiment 128 or 129, wherein the blocking agent is BSA.

131. The method according to any one of embodiments 129 or 130, wherein the one or more chaotropic and/or denaturing agents is a guanidine salt, for example guanidine hydrochloride.

132. The method according to any one of embodiments 124 to 131, wherein the components of the composition are provided so as to prepare a composition with the components and/or concentrations of any one of embodiments 1 to 122.

133. A method of carrying out an immunoassay, the method comprising contacting a biological sample with one or more compositions according to any one of embodiments 1 to 122.

134. The method of carrying out an immunoassay according to embodiment 133, wherein the immunoassay is for detecting presence of an analyte of interest in the biological sample.

135. The method of carrying out an immunoassay according to embodiment 133 or 134, wherein the biological sample is contacted with the one or more compositions according to any one of embodiments 1 to 122 simultaneously or sequentially.

136. The method of carrying out an immunoassay according to any one of embodiments 133 to 135, wherein the one or more compositions is used as a diluent for the biological sample.

137. The method of carrying out an immunoassay according to any one of embodiments 133 to 136, wherein the method comprises contacting the biological sample with a capture antibody, wherein the one or more compositions is used as a capture diluent when the biological sample is contacted with the capture antibody.

138. The method of carrying out an immunoassay according to any one of embodiments 133 to 137, wherein the method comprises addition of a detection antibody and the one or more compositions is used as a detection diluent when the detection antibody is added.

139. The method of carrying out an immunoassay according to embodiment 138, wherein addition of the detection antibody may occur simultaneously with or subsequently to contacting of the biological sample with the capture antibody.

140. The method of carrying out an immunoassay according to embodiment 138 or 139, wherein a wash step using a washing solution occurs after contacting the biological sample with a capture antibody and the detection diluent is added to the capture antibody prior to or simultaneously with addition of the detection antibody.

141. The method of carrying out an immunoassay according to any one of embodiments 133 to 140, wherein the one or more compositions is used as a washing solution.

142. The method of carrying out an immunoassay according to embodiment 141, wherein the washing solution comprises or consists essentially of tris buffered saline and a block copolymer, as defined in any of the preceding embodiments.

143. The method of carrying out an immunoassay according to any one of embodiments 133 to 142, wherein the one or more compositions is used as a buffer.

144. The method according to any one of embodiments 133 to 143 wherein the biological sample is a blood sample, for example a whole blood sample or a blood fraction, amniotic fluid; aqueous humour; bile; blood plasma; breast milk; cerebrospinal fluid (CSF), endolymph, extracellular fluid, exudate, gastric acid, hemolymph, interstitial fluid, lymph, mucus, pericardial fluid, peritoneal fluid, perspiration (sweat), phlegm, pus, saliva, semen, synovial fluid, tears, urine, vaginal fluids, vomit, sputum, other biofluid or swab sample.

145. The method according to any one of embodiments 133 to 144, wherein the biological sample is a whole blood sample.

146. The method according to any one of embodiments 133 to 145 wherein the biological sample comprises an analyte of interest.

147. The method according to any one of embodiments 134 to 146 wherein the analyte of interest is a protein.

148. The method according to embodiment 147 wherein the analyte of interest is brain natriuretic peptide or N-terminal pro-BNP.

149. The method according to embodiment 147 wherein the analyte of interest is cardiac troponin or cardiac troponin subunit I (cTnl).

150. The method according to any one of embodiments 133 to 149, wherein the immunoassay is a competitive sandwich immunoassay.

151. The method according to any one of embodiments 133 to 150, wherein the immunoassay comprises obtaining an electrochemical measurement to detect an analyte of interest.

152. The method according to any one of embodiments 133 to 151, wherein the electrochemical measurement is an amperometric, voltametric, potentiometric, impedimetric, or electrochemical impedance spectroscopic measurement.

153. The method according to embodiment 152, wherein the electrochemical measurement is a chronoamperometric measurement.

154. The method according to any one of embodiments 133 to 153, wherein the method is for measuring an analyte of interest in a biological sample and comprises:
a. combining said biological sample with:
   a first antibody or antigen binding portion thereof capable of binding said analyte of interest;
      a second antibody or antigen binding portion thereof capable of binding said analyte of interest conjugated to an enzyme; and
   an assay diluent, wherein the assay diluent is a composition according to any one of embodiments 1 to 122;
to form an assay mixture;
b. contacting the assay mixture with a substrate for the enzyme, wherein the enzyme substrate is converted by the enzyme into an electroactive molecule; and
c. obtaining an electrochemical measurement to measure the analyte of interest.

155. The method according to any one of embodiments 133 to 154, wherein the method is for measuring an analyte of interest in a biological sample and comprises:
a. combining said biological sample with:
   magnetically susceptible beads conjugated to a first antibody or antigen binding portion thereof capable of binding said analyte of interest;
   a second antibody or antigen binding portion thereof capable of binding said analyte of interest conjugated to an enzyme; and
   an assay diluent, wherein the assay diluent is a composition according to any one of embodiments 1 to 122;
to form an assay mixture
b. retaining the magnetically susceptible beads on an electrode using a magnetic field;
c. contacting the magnetically susceptible beads with a substrate for the enzyme, wherein the enzyme substrate is converted by the enzyme into an electroactive molecule; and
d. obtaining an electrochemical measurement using said electrode.

156. The method according to any one of embodiments 133 to 155, wherein the method is for measuring an analyte of interest in a biological sample and comprises:
a. combining said biological sample with magnetically susceptible beads conjugated to a first antibody or antigen binding portion thereof capable of binding said analyte of interest in the presence of a capture diluent, wherein the capture diluent is a composition according to any one of embodiments 1 to 122;
b. immobilizing the magnetically susceptible beads on a magnet and washing the beads;
c. providing a second antibody or antigen binding portion thereof capable of binding said analyte of interest conjugated to an enzyme in a detection diluent, wherein the detection diluent is a composition according to any one of embodiments 1 to 122 to produce a detection solution;
d. adding the detection solution to the washed magnetically susceptible beads;
e. washing the magnetically susceptible beads;
f. contacting the magnetically susceptible beads with a substrate for the enzyme, wherein the enzyme substrate is converted by the enzyme into an electroactive molecule; and
g. obtaining an electrochemical measurement to measure the analyte of interest.

157. The method according to embodiment 156, wherein washing in steps b. and e. is carried out with a washing solution, optionally wherein the washing solution is a composition according to any one of embodiments 1 to 122.

158. The method according to embodiment 157, wherein the washing solution comprises or consists essentially of tris buffered saline and a block copolymer, as defined in any of the preceding embodiments.

159. The method according to any one of embodiments 137 to 158, wherein the capture diluent is a composition according to any one of embodiments 1 to 122, comprising a buffer compound, at least one salt, one or more block copolymers, one or more blocking agents and one or more chaotropic and/or denaturing agents.

160. The method according to embodiment 159, wherein the capture diluent comprises at least two salts.

161. The method according to embodiment 159 or 160, wherein the capture diluent comprises tris, NaCl, CaCl₂), a polyoxyethylene-polyoxypropylene block copolymer (such as Synperonic® F 108), bovine serum albumin and guanidine hydrochloride (guanidine HCl).

162. The method according to any one of embodiments 138 to 161, wherein the detection diluent is a composition according to any one of embodiments 1 to 122, comprising a buffer compound, at least one salt, one or more block copolymers and one or more blocking agents.

163. The method according to embodiment 162, wherein the detection diluent comprises at least two salts.

164. The method according to embodiment 162 or 163, wherein the detection diluent comprises tris, NaCl, CaCl₂), a polyoxyethylene-polyoxypropylene block copolymer (such as Synperonic® F 108) and bovine serum albumin.

165. The method according to any one of embodiments 133 to 164, wherein the one or more compositions according to any one of embodiments 1 to 122 are prepared by a method according to any one of embodiments 124 to 132.

166. The method according to any one of embodiments 133 to 165, wherein the immunoassay is performed on a microfluidic cartridge, optionally a microfluidic disposable cartridge.

167. The method according to embodiment 166, wherein the one or more compositions according to any one of embodiments 1 to 122 are prepared by a method according to any one of embodiments 124 to 132 on the microfluidic cartridge.

168. The method according to embodiment 167, wherein the one or more compositions prepared by a method according to any one of embodiments 124 to 132 on the microfluidic cartridge are a capture diluent and/or a detection diluent.

169. Use of a composition according to any one of embodiments 1 to 123 in a point of care diagnostic assay, such as an immunoassay.

170. Use of a composition according to any one of embodiments 1 to 123 in an immunoassay.

171. The use according to embodiment 169 or 170, wherein the immunoassay is a competitive sandwich immunoassay.

172. The use according to any one of embodiments 169 to 171, wherein the immunoassay comprises obtaining an electrochemical measurement to detect an analyte of interest.

173. The use according to embodiment 172, wherein the electrochemical measurement is an amperometric, voltametric, potentiometric, impedimetric, or electrochemical impedance spectroscopic measurement.

174. The use according to embodiment 173, wherein the electrochemical measurement is a chronoamperometric measurement.

175. A kit comprising:
one or more compositions according to any one of embodiments 1 to 123; and
a point-of-care immunoassay diagnostic test.

176. The kit according to embodiment 175 further comprising instructional material, optionally wherein the instructional material comprises instructions for conducting a method of carrying out an immunoassay according to any one of embodiments 133 to 168.

### REFERENCES

[1] H. H. Bearat, B. L. Vernon, 11-Environmentally responsive injectable materials, Injectable Biomaterials, 2011, Pages 263-297, ISBN 9781845695880, incorporated herein by reference

1. A composition for immunoassays comprising a buffer compound, a salt and one or more block copolymers.

2. The composition for immunoassays according to claim 1, wherein the buffer compound is at a concentration of about 1 mM to about 500 mM.

3. The composition for immunoassays according to any preceding claim, comprising Tris (tris(hydroxymethyl) aminomethane or 2-amino-2-(hydroxymethyl) propane-1,3-diol) at a concentration of about 1 mM to about 100 mM.

4. The composition for immunoassays according to any preceding claim, wherein the salt is at a concentration of about 1 mM to about 1000 mM, optionally wherein the salt is sodium chloride (NaCl) at a concentration of about 50 to about 500 mM.

5. The composition for immunoassays according to any preceding claim, comprising one or more block copolymers at a total concentration of about 0.01% (w/v) to about 2% (w/v).

6. The composition for immunoassays according to any preceding claim, comprising Tris (tris(hydroxymethyl) aminomethane or 2-amino-2-(hydroxymethyl) propane-1,3-diol) at a concentration of about 1 mM to about 100 mM, sodium chloride (NaCl) at a concentration of about 1 mM to about 500 mM and one or more block copolymers at a total concentration of about 0.01% (w/v) to about 1% (w/v).

7. A composition for immunoassays comprising a buffer compound, one or more salts, one or more blocking agents and one or more block copolymers.

8. A composition for immunoassays according to claim 7, wherein the one or more blocking agents are selected from the list consisting of: proteins, such as phosphoproteins, polymers and small molecules.

9. A composition for immunoassays according to claim 8, wherein:
a. the protein is a phosphoprotein, optionally wherein the phosphoprotein is casein; or
b. the protein is bovine serum albumin (BSA).

10. A composition for immunoassays according to any one of claims 7 to 9 comprising at least two salts, for example two, three, four, five or six salts.

11. The composition for immunoassays according to any one of claims 7 to 10, wherein the buffer compound is at a concentration of about 1 mM to about 500 mM.

12. The composition for immunoassays according to any one of claims 7 to 11, comprising Tris (tris(hydroxymethyl) aminomethane or 2-amino-2-(hydroxymethyl) propane-1,3-diol) at a concentration of about 1 mM to about 100 mM.

13. The composition for immunoassays according to any one of claims 7 to 12, wherein one of the one or more salts is at a concentration of about 1 mM to about 1000 mM, optionally wherein the composition comprises sodium chloride (NaCl) at a concentration of:
a. about 300 mM to about 500 mM; or
b. about 100 mM to about 200 mM.

14. The composition for immunoassays according to any one of claims 7 to 13, further comprising calcium chloride (CaCl₂)) at a concentration of about 1 mM to about 200 mM.

15. The composition for immunoassays according to any one of claims 7 to 14, comprising one or more blocking agents at a total concentration of about 0.05% (w/v) to about 10% (w/v), for example about 0.05% (w/v) to about 9% (w/v), about 0.05% (w/v) to about 8% (w/v), about 0.05% (w/v) to about 7% (w/v), about 0.05% (w/v) to about 6% (w/v), about 0.05% (w/v) to about 5% (w/v), about 0.05% (w/v) to about 4% (w/v), about 0.05% (w/v) to about 3% (w/v), about 0.05% (w/v) to about 2.5% (w/v), about 0.05% (w/v) to about 2% (w/v), about 0.05% (w/v) to about 1.5% (w/v), about 0.05% (w/v) to about 1% (w/v), about 0.05% (w/v) to about 0.5% (w/v), about 0.05% (w/v) to about 0.4% (w/v), about 0.05% (w/v) to about 0.3% (w/v), about 0.05% (w/v) to about 0.2% (w/v) or about 0.05% (w/v) to about 0.1% (w/v).

16. The composition for immunoassays according to claim 15, comprising a blocking agent comprising bovine serum albumin at a concentration of about 0.05% (w/v) to about 10% (w/v).

17. The composition for immunoassays according to any one of claims 7 to 16, comprising Tris (tris(hydroxymethyl) aminomethane or 2-amino-2-(hydroxymethyl) propane-1,3-diol) at a concentration of about 1 mM to about 100 mM, sodium chloride (NaCl) at a concentration of about 100 mM to about 800 mM, a blocking agent comprising bovine serum albumin at a concentration of about 0.05% (w/v) to about 10% (w/v) and one or more block copolymers at a total concentration of about 0.01% (w/v) to about 1% (w/v).

18. The composition for immunoassays according to any one of claims 8 to 15, comprising casein at a concentration of about 0.1% (w/v) to about 3% (w/v).

19. The composition for immunoassays according to claim 18, comprising Tris (tris(hydroxymethyl) aminomethane or 2-amino-2-(hydroxymethyl) propane-1,3-diol) at a concentration of about 1 mM to about 100 mM, sodium chloride (NaCl) at a concentration of about 1 mM to about 500 mM, casein at a concentration of about 0.1% (w/v) to about 3% (w/v) and one or more block copolymers at a total concentration of about 0.05% (w/v) to about 2% (w/v).

20. The composition for immunoassays according to any preceding claim, wherein the block copolymer is a polyoxyethylene-polyoxypropylene block copolymer.

21. The composition for immunoassays according to any preceding claim further comprising one or more chaotropic and/or denaturing agents.

22. The composition for immunoassays according to claim 21, wherein the one or more chaotropic and/or denaturing agents are selected from a list consisting of: a guanidine salt, urea or betaine.

23. The composition for immunoassays according to claim 21 or claim 22, wherein the one or more chaotropic and/or denaturing agents is guanidine hydrochloride.

24. The composition for immunoassays according to any one of claims 21 to 23 wherein the one or more chaotropic and/or denaturing agents are at a concentration of about 1 mM to about 1000 mM.

25. The composition for immunoassays according to any preceding claim, wherein the composition is a liquid, a gel or an emulsion.
